SEQUENCE LISTING (10 sequences)

SEQ ID NO: 1 - protein, 1965 aa, synthetic construct
  REGION 1..1965: note = rFVIIIFc-XTEN amino acid sequence, including signal peptide
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPASSEITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - DNA, 5898 bp, synthetic construct
  misc_feature 1..5898: note = rFVIIIFc-XTEN nucleotide sequence
  source 1..5898: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctctcaaaacggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccagggtccccagccggcagtcccactagcaccgaggagggaacctctgaaagcgccacacccgaatcagggccagggtctgagcctgctaccagcggcagcgagacaccaggcacctctgagtccgccacaccagagtccggacccggatctcccgctgggagccccacctccactgaggagggatctcctgctggctctccaacatctactgaggaaggaacctcaaccgagccatccgagggatcagctcccggcacctcagagtcggcaaccccggagtctggacccggaacttccgaaagtgccacaccagagtccggtcccgggacttcagaatcagcaacacccgagtccggccctgggtctgaacccgccacaagtggtagtgagacaccaggatcagaacctgctacctcagggtcagagacacccggatctccggcaggctcaccaacctccactgaggagggcaccagcacagaaccaagcgagggctccgcacccggaacaagcactgaacccagtgagggttcagcacccggctctgagccggccacaagtggcagtgagacacccggcacttcagagagtgccacccccgagagtggcccaggcactagtaccgagccctctgaaggcagtgcgccagcctcgagcgaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctacgacaaaactcacacatgcccaccgtgcccagctccagaactcctgggcggaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaagtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaagttagcctgacctgcctggtcaaaggcttctatcccagcgacatcgccgtggagtgggagagcaatgggcagccggagaacaactacaagaccacgcctcccgtgttggactccgacggctccttcttcctctactccaagctcaccgtggacaagagcaggtggcagcaggggaacgtcttctcatgctccgtgatgcatgaggctctgcacaaccactacacgcagaagagcctctccctgtctccgggttga

SEQ ID NO: 3 - protein, 1946 aa, synthetic construct
  REGION 1..1946: note = rFVIIIFc-XTEN amino acid sequence without signal peptide
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPASSEITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 1646 aa, synthetic construct
  REGION 1..1646: note = rVWFFc-XTEN amino acid sequence with signal peptide and D1D2portion of VWF
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDIQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCAAFCDTIAAYAHVCAQHGKVVTWRTATLCPQSCEERNLRENGYEAEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGASSDKNTGDYYEDSYEDISAYLLSKNNAIEPRSFSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - DNA, 4941 bp, synthetic construct
  misc_feature 1..4941: note = rVWFFc-XTEN nucleotide sequence
  source 1..4941: mol_type = other DNA
atgattcctgccagatttgccggggtgctgcttgctctggccctcattttgccagggaccctttgtgcagaaggaactcgcggcaggtcatccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcactggtcaatcccacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacatccagctccccctcctgaaaggtgacctccgcatccagcatacagtgacggcctccgtgcgcctcagctacggggaggacctgcagatggactgggatggccgcgggaggctgctggtgaagctgtcccccgtctatgccgggaagacctgcggcctgtgtgggaattacaatggcaaccagggcgacgacttccttaccccctctgggctggcggagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgccgcattctgcgacaccattgctgcctatgcccacgtgtgtgcccagcatggcaaggtggtgacctggaggacggccacattgtgcccccagagctgcgaggagaggaatctccgggagaacgggtatgaggctgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggccggcgttttgcctcaggaaagaaagtcaccttgaatcccagtgaccctgagcactgccagatttgccactgtgatgttgtcaacctcacctgtgaagcctgccaggagccgggtacatcagagagcgccacccctgaaagtggtcccgggagcgagccagccacatctgggtcggaaacgccaggcacatccgagtctgcaactcccgagtccggacctggctccgagcctgccactagcggctccgagactccgggaacttccgagagcgctacaccagaaagcggacccggaaccagtaccgaacctagcgagggctctgctccgggcagcccagccggctctcctacatccacggaggagggcacttccgaatccgccaccccggagtcagggccaggatctgaacccgctacctcaggcagtgagacgccaggaacgagcgagtccgctacaccggagagtgggccagggagccctgctggatctcctacgtccactgaggaagggtcaccagcgggctcgcccaccagcactgaagaaggtgcctcgtctgacaagaacactggtgattattacgaggacagttatgaagatatttcagcatacttgctgagtaaaaacaatgccattgaaccaagaagcttctctgacaaaactcacacatgcccaccgtgcccagctccagaactcctgggcggaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaagtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaagttagcctgacctgcctggtcaaaggcttctatcccagcgacatcgccgtggagtgggagagcaatgggcagccggagaacaactacaagaccacgcctcccgtgttggactccgacggctccttcttcctctactccaagctcaccgtggacaagagcaggtggcagcaggggaacgtcttctcatgctccgtgatgcatgaggctctgcacaaccactacacgcagaagagcctctccctgtctccgggttga

SEQ ID NO: 6 - protein, 883 aa, synthetic construct
  REGION 1..883: note = rVWFFc-XTEN amino acid sequence without signal peptide or D1D2portion of VWF
SLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCAAFCDTIAAYAHVCAQHGKVVTWRTATLCPQSCEERNLRENGYEAEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGASSDKNTGDYYEDSYEDISAYLLSKNNAIEPRSFSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 2332 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 8 - protein, 288 aa, synthetic construct
  REGION 1..288: note = Synthesized
GTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 9 - protein, 144 aa, synthetic construct
  REGION 1..144: note = Synthesized
TSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEG

SEQ ID NO: 10 - protein, 32 aa, synthetic construct
  REGION 1..32: note = Synthesized
DKNTGDYYEDSYEDISAYLLSKNNAIEPRSFS

(19) United States
(12) Patent Application Publication
(10) Pub. No.: US 2025/0273317 A1
(43) Pub. Date: Aug. 28, 2025

(54) METHODS FOR TREATING HEMOPHILIA A AND POPULATION PHARMACOKINETICS TOOLS FOR DETERMINING TREATMENTS AND USES THEREOF

(71) Applicant: BIOVERATIV THERAPEUTICS INC., Waltham, MA (US)

(72) Inventors: Pratik BHAGUNDE, New Providence, NJ (US); Suresh KATRAGADDA, Braintree, MA (US)

(21) Appl. No.: 19/040,048

(22) Filed: Jan. 29, 2025

Related U.S. Application Data
(63) Continuation of application No. PCT/US2023/071400, filed on Aug. 1, 2023.
(60) Provisional application No. 63/485,418, filed on Feb. 16, 2023.
(60) Provisional application No. 63/383,091, filed on Nov. 10, 2022.
(60) Provisional application No. 63/370,010, filed on Aug. 1, 2022.

(51) Int. Cl. G16H 20/17; A61K 38/37; G16H 10/60

(52) U.S. Cl. CPC G16H 20/17; A61K 38/37; G16H 10/60

(57) ABSTRACT

The present disclosure provides, inter alia, methods for treating hemophilia A as well as computer-based-systems, including software-based pharmacokinetic model systems, and their use to determine dosing information for subjects who have hemophilia A.

### CROSS-REFERENCE TO RELATED APPLICATIONS

This application is a continuation of International Patent Application No. PCT/US2023/071400, filed Aug. 1, 2023, which claims the benefit of U.S. Provisional Application No. 63/370,010, filed Aug. 1, 2022; U.S. Provisional Application No. 63/383,091, filed Nov. 10, 2022; and U.S. Provisional Application No. 63/485,418, filed Feb. 16, 2023; each of which is incorporated by reference in its entirety for all purposes.

### REFERENCE TO SEQUENCE LISTING SUBMITTED ELECTRONICALLY

The content of the electronically submitted sequence listing in XML file (Name: 760421_SA9-488PCCON_ST26.xml; Size: 33,379 bytes; Date of Creation: Jan. 27, 2025) is incorporated herein by reference in its entirety.

### BACKGROUND

While plasma-derived and recombinant clotting factor products allow hemophilia patients to live longer and healthier lives, hemophilia remains one of the most costly and complex conditions to manage. Due to its complexity, treatment of hemophilia A using FVIII replacement therapy requires a special therapeutic management process for doctors, pharmacies, and patients. Clinicians often assess lifestyle, psychosocial requirements, and the home environment when evaluating a patient's or guardian's ability to provide adequate care.

The current recommended standard of care involves the regular administration (routine prophylaxis) of FVIII to minimize the number of bleeding episodes. Routine prophylaxis has been associated with improvements in long-term outcomes, but is a demanding regimen limited by the need for frequent intravenous (IV) administration. See Manco-Johnson et al., N Engl J Med. 357 (6): 535-44 (2007). Extended half-life FVIII products have reduced the frequency of FVIII administration for prophylaxis; however, currently available FVIII products that interact with endogenous von Willebrand factor (VWF) have comparable circulating half-lives, consistent with an upper limit on the half-life of rFVIII variants due to the half-life of endogenous VWF. See, e.g., Pipe et al., Blood. 128 (16): 2007-16 (2016).

### BRIEF SUMMARY

Provided herein are, inter alia, methods of treating hemophilia A, as well as software-based pharmacokinetics systems and their use to provide dosing information (such as a dose and a dosing interval) for a subject in need of treatment for hemophilia A.

In some aspects, the present disclosure comprises a method (e.g., a computer-implemented method) of determining (e.g., calculating, estimating, or providing) efanesoctocog alfa dosing information for an individual subject. In some embodiments, the method comprises receiving information specific to the subject and calculating the efanesoctocog alfa dosing information using a software-based system, wherein the system is programmed to implement a one-compartment efanesoctocog alfa popPK model, wherein the efanesoctocog alfa popPK model comprises body weight as a covariate, and wherein the efanesoctocog alfa popPK model does not comprise the level of von Willebrand factor (VWF) or level of hematocrit as covariates. In some embodiments, the method further comprises outputting, by the software-based system, the dosing information for the subject. In some embodiments, the method further comprises outputting, by the software-based system, a suggested dose regimen. In some embodiments, desired treatment outcome information is also received.

In some aspects, the present disclosure comprises a method (e.g., a computer-implemented method) of estimating (e.g., calculating, determining, or providing) individualized efanesoctocog alfa dosing information for a subject, the method comprising: (a) receiving, by a processing device, subject information and/or desired treatment outcome information by an application program programmed to operate with an efanesoctocog alfa popPK model, wherein the received information is transmitted by one or more electronic devices, (b) calculating, using at least a software-based system, individualized efanesoctocog alfa dosing information using the efanesoctocog alfa popPK model and the received information, and (c) transmitting the calculated individualized efanesoctocog alfa dosing information of (b) to one or more electronic devices for output of the information, wherein the efanesoctocog alfa popPK model comprises body weight as a covariate, and wherein the efanesoctocog alfa popPK model does not comprise the level of VWF or the level of hematocrit as covariates.

In some aspects, the present disclosure comprises a method (e.g., a computer-implemented method) of estimating (e.g., calculating, determining, or providing) individualized efanesoctocog alfa dosing information for a subject, the method comprising: (a) receiving subject information and/or desired treatment outcome information by one or more electronic devices, (b) transmitting, by a processing device, the subject information and/or desired treatment outcome information to an application program, wherein the application is programmed to implement an efanesoctocog alfa popPK model, (c) receiving from the application program, individualized efanesoctocog alfa dosing information calculated using the efanesoctocog alfa popPK model and the transmitted information of (b), and (d) outputting, by the one or more electronic devices, the individualized efanesoctocog alfa dosing information, wherein the efanesoctocog alfa popPK model comprises body weight as a covariate, and wherein the efanesoctocog alfa popPK model does not comprise the level of VWF or level of hematocrit as covariates.

In some aspects, the present disclosure comprises a method (e.g., a computer-implemented method) of providing (e.g., calculating, determining, or estimating) an efanesoctocog alfa dosing regimen based on median popPK, the method comprising: (a) receiving subject information and/or desired treatment outcome information by a software-based system comprising an efanesoctocog alfa popPK model, (b) calculating, by the software-based system, median PK information using the efanesoctocog alfa popPK model and the received information, and (c) outputting, by the software-based system, the median PK information, wherein the efanesoctocog alfa popPK model comprises body weight as a covariate, and wherein the efanesoctocog alfa popPK model does not comprise the level of VWF or level of hematocrit as covariates.

In some aspects, the present disclosure comprises a method (e.g., a computer-implemented method) of providing (e.g., calculating, determining, or estimating) an efanesoctocog alfa dosing regimen based on median efanesoctocog alfa popPK, the method comprising: (a) receiving subject information and/or desired treatment outcome information by one or more electronic devices, (b) transmitting, by a processing device, the subject information and/or desired treatment outcome information to an application program, wherein the application is programmed to implement an efanesoctocog alfa popPK model, (c) receiving from the application program, median efanesoctocog alfa PK dosing information calculated using the efanesoctocog alfa popPK model and the received information, and (d) outputting, by the one or more electronic devices, the median PK information, wherein the efanesoctocog alfa popPK model comprises body weight as a covariate, and wherein the efanesoctocog alfa popPK model does not comprise the level of VWF or level of hematocrit as covariates.

In some aspects, the present disclosure comprises a method (e.g., a computer-implemented method) of providing (e.g., calculating, determining, or estimating) an efanesoctocog alfa dosing regimen, the method comprising: (a) receiving, by a processing device, subject information and/or desired treatment outcome information by an application program programmed to implement an efanesoctocog alfa population pharmacokinetic (popPK) model, wherein the received information is transmitted by one or more electronic devices, (b) calculating, by the application program, individualized efanesoctocog alfa dosing information using the efanesoctocog alfa popPK model and the received information, and (c) transmitting, by a processing device, the individualized efanesoctocog alfa dosing regimen calculated dosing information of (b) to one or more electronic devices for output of the information, wherein the efanesoctocog alfa popPK model comprises body weight as a covariate, and wherein the efanesoctocog alfa popPK model does not comprise the level of VWF or level of hematocrit as covariates.

In some aspects, the present disclosure comprises a method (e.g., a computer-implemented method) of estimating (e.g., calculating, determining, or providing) individualized subject efanesoctocog alfa PK information, the method comprising: (a) receiving subject information by a software-based system comprising an efanesoctocog alfa popPK model, (b) estimating, by the software-based system, individualized subject efanesoctocog alfa PK information using the efanesoctocog alfa popPK model and the received information, and (c) outputting, by the software-based system, the individualized subject efanesoctocog alfa PK information, wherein the efanesoctocog alfa popPK model comprises body weight as a covariate, and wherein the efanesoctocog alfa popPK model does not comprise the level of VWF or level of hematocrit as covariates.

In some aspects, the present disclosure comprises a method (e.g., a computer-implemented method) of estimating (e.g., calculating, determining, or providing) individualized subject efanesoctocog alfa PK information, the method comprising: (a) receiving, by one or more electronic devices, subject information, (b) transmitting, by a processing device, the subject information to an application program, wherein the application is programmed to implement an efanesoctocog alfa popPK model, and wherein the application program generates individualized subject efanesoctocog alfa PK information using the efanesoctocog alfa popPK model and the transmitted information, (c) receiving the individualized subject efanesoctocog alfa PK information from the application program, and (d) outputting, by the one or more electronic devices, the individualized subject PK information, wherein the efanesoctocog alfa popPK model comprises body weight as a covariate, and wherein the efanesoctocog alfa popPK model does not comprise the level of VWF or level of hematocrit as covariates.

In some aspects, the present disclosure comprises a method (e.g., a computer-implemented method) of estimating (e.g., calculating, determining, or providing) individualized subject efanesoctocog alfa PK information, the method comprising: (a) receiving subject information by an application program that is programmed to implement an efanesoctocog alfa popPK model, wherein the received information is transmitted by one or more electronic devices, (b) calculating, by the application program, individualized subject efanesoctocog alfa PK information of efanesoctocog alfa using the efanesoctocog alfa popPK model and the received information, and (c) transmitting, by a processing device, the calculated individualized subject efanesoctocog alfa PK information of (b) to one or more one or more electronic devices, for output of the information, wherein the efanesoctocog alfa popPK model comprises body weight as a covariate, and wherein the efanesoctocog alfa popPK model does not comprise the level of VWF or level of hematocrit as covariates.

In some aspects, the present disclosure comprises a method (e.g., a computer-implemented method) of estimating (e.g., calculating, determining, or providing) individualized subject efanesoctocog alfa PK information, the method comprising: (a) receiving, by one or more electronic devices, information regarding individual body weight and (i) desired raise of plasma factor activity level following the dose or (ii) desired dose or desired dose interval, (b) transmitting, by a processing device, the information of (a) to an application program, wherein the application is programmed to implement an efanesoctocog alfa popPK model, (c) receiving from the web-based server and program, individualized subject efanesoctocog alfa PK information calculated using the efanesoctocog alfa popPK model and the transmitted information of (b), and (d) outputting, by the one or more electronic devices, the calculated individualized subject efanesoctocog alfa PK information, wherein the efanesoctocog alfa popPK model comprises body weight as a covariate, and wherein the efanesoctocog alfa popPK model does not comprise the level of VWF or level of hematocrit as covariates.

In some embodiments of the methods above, the subject information includes the subject's body weight.

In some embodiments of the methods above, the subject information includes a baseline FVIII activity level for the subject.

In some embodiments of the methods above, the subject information includes the subject's self-reported race. In some embodiments of the methods above, the subject information includes whether the subject self-identifies as being Asian.

In some embodiments of the methods above, the subject provides the subject information. In some embodiments of the methods above, a healthcare professional provides the subject information.

In some embodiments of the methods above, the subject information does not include the subject's level of VWF or hematocrit.

In some embodiments of the methods above, the system is programmed to implement a one-compartment efanesoctocog alfa popPK model comprising body weight as covariates to calculate the dosing information, and wherein the efanesoctocog alfa popPK model does not comprise the level of VWF or hematocrit as covariates.

In some embodiments of the methods above, the method also includes selecting a dosing regimen based on the dosing information.

In some embodiments of the methods above, the method also includes selecting a dosing regimen based on the PK information.

In some embodiments of the methods above, the method also includes administering the efanesoctocog alfa to the subject according to the selected dosing regimen.

In some embodiments of the methods above, the dosing information comprises estimated or predicted FVIII activity levels over time after administration of the efanesoctocog alfa.

In some embodiments of the methods above, the dosing regimen is a prophylaxis regimen. In some embodiments of the methods above, the dosing regimen is an on-demand regimen. In some embodiments of the methods above, the dosing regimen is for perioperative management of bleeding.

In some embodiments of the methods above, the desired treatment outcome information comprises a desired FVIII activity level. In some embodiments of the methods above, the desired FVIII activity level comprises the minimum FVIII activity level between doses.

In some embodiments of the methods above, the desired FVIII activity level comprises the minimum FVIII activity level at a time point. In some embodiments of the methods above, the time point is 1, 2, 3, 4, 5, 6, 7, 8, 9, 10, 11, 12, 13, or 14 days after an administration of efanesoctocog alfa. In some embodiments, the time point is about 1 week after an administration of efanesoctocog alfa.

In some embodiments of the methods above, the efanesoctocog alfa popPK model comprises self-reported race as a covariate. In some embodiments of the methods above, the efanesoctocog alfa popPK model comprises whether the subject self-reports as Asian as a covariate.

In some embodiments of the methods above, the subject does not self-report as being Asian. In some embodiments of the methods above, the subject self-reports as being Asian.

In some embodiments of the methods above, the efanesoctocog alfa popPK model is the efanesoctocog alfa popPK model [A].

In some embodiments, the estimate of typical clearance (TVCL) in the efanesoctocog alfa popPK model [A] is about 0.433 dL/h, the estimate of typical volume (TVV) in the -5-fanesoctocog alfa popPK model [A] is about 30.2 dL, the variability on clearance from central compartment (η1) in the efanesoctocog alfa popPK model [A] is about 0.0354, and/or the variability on volume of central compartment (η2) in the efanesoctocog alfa popPK model [A] is about 0.0209. In some embodiments, the estimate of typical clearance (TVCL) in the efanesoctocog alfa popPK model [A] is about 0.433 dL/h, the estimate of typical volume (TVV) in the -6-fanesoctocog alfa popPK model [A] is about 30.2 dL, the variability on clearance from central compartment (η1) in the efanesoctocog alfa popPK model [A] is about 0.0354, and the variability on volume of central compartment (η2) in the efanesoctocog alfa popPK model [A] is about 0.0209. In some embodiments, the estimate of typical clearance (TVCL) in the efanesoctocog alfa popPK model [A] is 0.433 dL/h, the estimate of typical volume (TVV) in the efanesoctocog alfa popPK model [A] is 30.2 dL, the variability on clearance from central compartment (η1) in the efanesoctocog alfa popPK model [A] is 0.0354, and the variability on volume of central compartment (η2) in the efanesoctocog alfa popPK model [A] is 0.0209.

In some embodiments of the methods disclosed herein, the electronic device is a digital pen, a smart phone, a tablet computer, a personal digital assistant, a handheld computer, a laptop computer, a scanner, a camera, and/or a fax machine.

Also included is a method (e.g., a computer-implemented method) off treating hemophilia A in a subject in need thereof, comprising administering to the subject a dose regimen selected according to any one of the methods above.

Also disclosed is a system comprising a processor configured to provide dosing or PK information according to the method any one of the methods above. In some embodiments, the system is a network-based system. In some embodiments, the system is a web-based system.

In some embodiments, the system is programmed to implement a one-compartment efanesoctocog alfa popPK model with linear elimination comprising body weight as covariates to calculate the dosing information, and wherein the efanesoctocog alfa popPK model does not comprise the level of VWF or hematocrit as covariates.

In some embodiments, the system is programmed to implement a one-compartment efanesoctocog alfa popPK model with linear elimination comprising body weight as covariates to calculate the individualized subject efanesoctocog alfa PK information, and wherein the efanesoctocog alfa popPK model does not comprise the level of VWF or hematocrit as covariates.

In some aspects, the present disclosure comprises a data processing apparatus, device, or system comprising a processor configured to implement a one-compartment efanesoctocog alfa popPK model, wherein the efanesoctocog alfa popPK model comprises body weight as a covariate, and wherein the efanesoctocog alfa popPK model does not comprise the level of VWF or level of hematocrit as covariates.

In some embodiments of the data processing apparatus, device, or system above, the efanesoctocog alfa popPK model comprises self-reported race as a covariate.

In some embodiments of the data processing apparatus, device, or system above, the efanesoctocog alfa popPK model comprises whether the subject self-reports as Asian as a covariate.

In some embodiments of any data processing apparatus, device, or system above, the data processing apparatus, device, or system comprises a smart phone, a tablet computer, a personal digital assistant, a handheld computer, a laptop computer, or a smart watch.

In some embodiments of any data processing apparatus, device, or system above, the data processing apparatus, device, or system comprises a smart phone. In some embodiments of any data processing apparatus, device, or system above, the data processing apparatus, device, or system comprises a smart watch.

In some embodiments of any data processing apparatus, device, or system above, the data processing apparatus, device, or system the processor is configured to implement efanesoctocog alfa popPK model [A].

Also disclosed is a computer program comprising instructions which, when the program is executed by a computer, cause the computer to carry out any one of the methods above.

In some aspects, the present disclosure comprises a computer program comprising instructions which, when the program is executed by a computer, cause the computer to implement a one-compartment efanesoctocog alfa popPK model, wherein the efanesoctocog alfa popPK model comprises body weight as a covariate, and wherein the efanesoctocog alfa popPK model does not comprise the level of VWF or level of hematocrit as covariates.

In some embodiments of the computer program above, the efanesoctocog alfa popPK model comprises self-reported race as a covariate. In some embodiments of the computer program above, the efanesoctocog alfa popPK model comprises whether the subject self-reports as Asian as a covariate.

In some embodiments of the computer program above, the efanesoctocog alfa popPK model is efanesoctocog alfa popPK model [A].

In some embodiments, the computer program is accessible through a web server or network server.

Also disclosed is a computer-readable medium comprising instructions which, when executed by a computer, cause the computer to provide carry out any one of the methods above.

In some aspects, the present disclosure comprises a computer-readable medium comprising instructions which, when executed by a computer, cause the computer to implement a one-compartment efanesoctocog alfa popPK model, wherein the efanesoctocog alfa popPK model comprises body weight as a covariate, and wherein the efanesoctocog alfa popPK model does not comprise the level of VWF or level of hematocrit as covariates.

In some embodiments of the computer-readable medium above, the efanesoctocog alfa popPK model comprises self-reported race as a covariate. In some embodiments of the computer-readable medium above, the efanesoctocog alfa popPK model comprises whether the subject self-reports as Asian as a covariate.

In some embodiments of the computer-readable medium above, the efanesoctocog alfa popPK model is efanesoctocog alfa popPK model [A].

In some embodiments, the computer-readable medium is accessible through a web server or network server.

Included herein is a method for determining (e.g., calculating estimating, or providing) chimeric protein dosing information for an individual subject, wherein the chimeric protein comprises (i) a factor VIII (FVIII) protein and (ii) a von Willebrand factor (VWF) fragment comprising a D′ domain of VWF and a D3 domain of VWF.

In some embodiments, the method comprises receiving information specific to the subject and calculating the chimeric protein dosing information using a software-based system, wherein the system is programmed to implement a one-compartment chimeric protein popPK model, wherein the chimeric protein popPK model comprises body weight as a covariate, and wherein the chimeric protein popPK model does not comprise the level of VWF or level of hematocrit as covariates.

In some embodiments, the method comprises outputting, by the software-based system, the dosing information for the subject. In some embodiments, the method comprises outputting, by the software-based system, a suggested dose regimen. In some embodiments, desired treatment outcome information is also received.

The present disclosure provides a method of estimating individualized chimeric protein dosing information for a subject, wherein the chimeric protein comprises (i) a factor VIII (FVIII) protein and (ii) a von Willebrand factor (VWF) fragment comprising a D′ domain of VWF and a D3 domain of VWF.

In some embodiments, the method comprises: (a) receiving, by a processing device, subject information and/or desired treatment outcome information by an application program programmed to operate with a chimeric protein popPK model, wherein the received information is transmitted by one or more electronic devices, (b) calculating, using at least a software-based system, individualized chimeric protein dosing information using the chimeric protein popPK model and the received information, and (c) transmitting the calculated individualized chimeric protein dosing information of (b) to one or more electronic devices for output of the information, wherein the chimeric protein popPK model comprises body weight as a covariate, and wherein the chimeric protein popPK model does not comprise the level of VWF or the level of hematocrit as covariates.

In some embodiments, the method comprises: (a) receiving subject information and/or desired treatment outcome information by one or more electronic devices, (b) transmitting, by a processing device, the subject information and/or desired treatment outcome information to an application program, wherein the application is programmed to implement a chimeric protein popPK model, (c) receiving from the application program, individualized chimeric protein dosing information calculated using the chimeric protein popPK model and the transmitted information of (b), and (d) outputting, by the one or more electronic devices, the individualized chimeric protein dosing information, wherein the chimeric protein popPK model comprises body weight as a covariate, and wherein the chimeric protein popPK model does not comprise the level of VWF or level of hematocrit as covariates.

Also included herein is method of estimating individualized subject chimeric protein PK information, wherein the chimeric protein comprises (i) a factor VIII (FVIII) protein and (ii) a von Willebrand factor (VWF) fragment comprising a D′ domain of VWF and a D3 domain of VWF.

In some embodiments, the method comprises: (a) receiving subject information by a software-based system comprising a chimeric protein popPK model, (b) estimating, by the software-based system, individualized subject chimeric protein PK information using the chimeric protein popPK model and the received information, and (c) outputting, by the software-based system, the individualized subject chimeric protein PK information, wherein the chimeric protein popPK model comprises body weight as a covariate, and wherein the chimeric protein popPK model does not comprise the level of VWF or level of hematocrit as covariates.

In some embodiments, the method comprises: (a) receiving, by one or more electronic devices, subject information, (b) transmitting, by a processing device, the subject information to an application program, wherein the application is programmed to implement a chimeric protein popPK model, and wherein the application program generates individualized subject chimeric protein PK information using the chimeric protein popPK model and the transmitted information, (c) receiving the individualized subject chimeric protein PK information from the application program, and (d) outputting, by the one or more electronic devices, the individualized subject PK information, wherein the chimeric protein popPK model comprises body weight as a covariate, and wherein the chimeric protein popPK model does not comprise the level of VWF or level of hematocrit as covariates.

In some embodiments, the method comprises: (a) receiving subject information by an application program that is programmed to implement a chimeric protein popPK model, wherein the received information is transmitted by one or more electronic devices, (b) calculating, by the application program, individualized subject chimeric protein PK information of chimeric protein using the chimeric protein popPK model and the received information, and (c) transmitting, by a processing device, the calculated individualized subject chimeric protein PK information of (b) to one or more one or more electronic devices, for output of the information, wherein the chimeric protein popPK model comprises body weight as a covariate, and wherein the chimeric protein popPK model does not comprise the level of VWF or level of hematocrit as covariates.

In some embodiments, the method comprising: (a) receiving, by one or more electronic devices, information regarding individual body weight and (i) desired raise of plasma factor activity level following the dose or (ii) desired dose or desired dose interval, (b) transmitting, by a processing device, the information of (a) to an application program, wherein the application is programmed to implement a chimeric protein popPK model, (c) receiving from the web-based server and program, individualized subject chimeric protein PK information calculated using the chimeric protein popPK model and the transmitted information of (b), and (d) outputting, by the one or more electronic devices, the calculated individualized subject chimeric protein PK information, wherein the chimeric protein popPK model comprises body weight as a covariate, and wherein the chimeric protein popPK model does not comprise the level of VWF or level of hematocrit as covariates.

In some embodiments, the subject information includes the subject's body weight.

In some embodiments, the subject information includes a baseline FVIII activity level for the subject.

In some embodiments, the subject information includes the subject's self-reported race. In some embodiments, the subject information includes whether the subject self-identifies as being Asian.

In some embodiments, the subject provides the subject information. In some embodiments, a healthcare professional provides the subject information.

In some embodiments, the subject information does not include the subject's level of VWF or hematocrit.

In some embodiments, the system is programmed to implement a one-compartment chimeric protein popPK model comprising body weight as covariates to calculate the dosing information, and wherein the chimeric protein popPK model does not comprise the level of VWF or hematocrit as covariates. In some embodiments, the system is programmed to implement a one-compartment chimeric protein popPK model with linear elimination comprising body weight as covariates to calculate the dosing information, and wherein the chimeric protein popPK model does not comprise the level of VWF or hematocrit as covariates. In some embodiments, the system is programmed to implement a one-compartment chimeric protein popPK model comprising body weight as covariates to calculate the individualized subject PK information, and wherein the chimeric protein popPK model does not comprise the level of VWF or hematocrit as covariates. In some embodiments, the system is programmed to implement a one-compartment chimeric protein popPK model with linear elimination comprising body weight as covariates to calculate the individualized subject PK information, and wherein the chimeric protein popPK model does not comprise the level of VWF or hematocrit as covariates.

In some embodiments, the method further comprises selecting a dosing regimen based on the dosing information. In some embodiments, the method further comprises selecting a dosing regimen based on the PK information. In some embodiments, the method further comprises administering the chimeric protein to the subject according to the selected dosing regimen.

In some embodiments, the dosing information comprises estimated or predicted FVIII activity levels over time after administration of the chimeric protein.

In some embodiments, the dosing regimen is a prophylaxis regimen. In some embodiments, the dosing regimen is an on-demand regimen. In some embodiments, the dosing regimen is for perioperative management of bleeding.

In some embodiments, the desired treatment outcome information comprises a desired FVIII activity level. In some embodiments, the desired FVIII activity level comprises the minimum FVIII activity level between doses. In some embodiments, the desired FVIII activity level comprises the minimum FVIII activity level at a time point. In some embodiments, the time point is 1, 2, 3, 4, 5, 6, 7, 8, 9, 10, 11, 12, 13, or 14 days after an administration of chimeric protein. In some embodiments, the time point is about 1 week after an administration of chimeric protein.

In some embodiments, the chimeric protein popPK model comprises self-reported race as a covariate. In some embodiments, the chimeric protein popPK model comprises whether the subject self-reports as Asian as a covariate. In some embodiments, the subject does not self-report as being Asian. In some embodiments, the subject self-reports as being Asian.

Included herein is a method of treating hemophilia A in a subject in need thereof, comprising administering to the subject a dose regimen selected according to a method disclosed herein.

Also provided is a device or system comprising a processor configured to provide dosing or PK information according to a method disclosed herein.

The present disclosure further provides a data processing apparatus, device, or system comprising a processor configured to implement a one-compartment chimeric protein popPK model, wherein the chimeric protein popPK model comprises body weight as a covariate, and wherein the chimeric protein popPK model does not comprise the level of VWF or level of hematocrit as covariates. In some embodiments, the chimeric protein popPK model comprises self-reported race as a covariate. In some embodiments, the chimeric protein popPK model comprises whether the subject self-reports as Asian as a covariate. In some embodiments, the data processing apparatus, device, or system comprises a smart phone, a tablet computer, a personal digital assistant, a handheld computer, a laptop computer, or a smart watch. In some embodiments, the data processing apparatus, device, or system comprises a smart phone and/or a smart watch.

Included herein is a computer program comprising instructions which, when the program is executed by a computer, cause the computer to carry out a method disclosed herein, e.g., a method that comprises a chimeric protein popPK model, wherein the chimeric protein popPK model comprises body weight as a covariate, but does not comprise the level of VWF or level of hematocrit as covariates. In some embodiments, the chimeric protein popPK model comprises self-reported race as a covariate. In some embodiments, the chimeric protein popPK model comprises whether the subject self-reports as Asian as a covariate.

Provided herein is a computer-readable medium comprising instructions which, when executed by a computer, cause the computer to provide carry out a method disclosed herein, e.g., a method that comprises a chimeric protein popPK model, wherein the chimeric protein popPK model comprises body weight as a covariate, but does not comprise the level of VWF or level of hematocrit as covariates. In some embodiments, the chimeric protein popPK model comprises self-reported race as a covariate. In some embodiments, the chimeric protein popPK model comprises whether the subject self-reports as Asian as a covariate.

In some embodiments, the FVIII polypeptide has a deletion of amino acids 746 to 1648 corresponding to mature FVIII (SEQ ID NO: 7) the first ELNN polypeptide is inserted within the FVIII polypeptide immediately downstream of amino acid 745 corresponding to mature FVIII (SEQ ID NO: 7) and the first ELNN polypeptide comprises an amino acid sequence that is least about 90% identical to the amino acid sequence of SEQ ID NO: 8.

In some embodiments, the VWF fragment comprises a D′ domain of VWF and a D3 domain of VWF, the VWF fragment is mutated to substitute cysteines involved in VWF dimerization to alanine, the second ELNN polypeptide comprises an amino acid sequence that is at least about 90% identical to the amino acid sequence of SEQ ID NO: 9, and the linker comprises an amino acid sequence that is at least about 90% identical to the amino acid sequence of SEQ ID NO: 10.

In some embodiments, the first ELNN polypeptide comprises the sequence of SEQ ID NO: 8 and the second ELNN polypeptide comprises the amino acid sequence of SEQ ID NO: 9.

In some embodiments, the chimeric protein comprises a first polypeptide and a second polypeptide, wherein first polypeptide comprises an amino acid sequence that is at least about 95% identical to the amino acid sequence of SEQ ID NO: 3 and the second polypeptide comprises an amino acid sequence that is at least about 95% identical to the amino acid sequence of SEQ ID NO: 6, and wherein the first polypeptide and the second polypeptide are covalently linked by two disulfide bonds between the first Fc region and the second Fc region.

In some embodiments, the chimeric protein comprises a first polypeptide and a second polypeptide, wherein the first polypeptide comprises the amino acid sequence set forth as SEQ ID NO: 3 and the second polypeptide comprises the amino acid sequence set forth as SEQ ID NO: 6, and wherein the first polypeptide and the second polypeptide are covalently linked by two disulfide bonds between the first Fc region and the second Fc region.

### BRIEF DESCRIPTION OF DRAWINGS/FIGURES

FIG. 1 is a visual representation of a software-based system that can be used in methods disclosed herein.

FIG. 2 is a visual representation of an exemplary network-based system that can be used according to the methods disclosed herein.

FIG. 3 shows a schematic diagram of an example computing system 400.

FIG. 4 is a graph showing efanesoctocog alfa individual clearance (dL/h) with baseline VWF levels (IU/dL) in adult and adolescent patients.

FIG. 5 is a graph showing simulated steady-state FVIII activity over time using efanesoctocog alfa population PK model [A] (50 IU/kg) in patients ≥12 years of age, based on clinical data derived from the one-stage clotting assay. Observed FVIII activity from the clinical data is also shown. Solid line is simulated median FVIII activity (IU/dL). Dashed lines are simulated 5th and 95th percentiles.

FIG. 6A is a graph showing a baseline corrected FVIII activity time profile from adult/adolescent study EFC16293, at day 1. Data is shown for all patients. LLOQ=1 IU/dL FIG. 6B is a graph showing a baseline corrected FVIII activity time profile from adult/adolescent study EFC16293, at day 1 (solid line) and week 26 (dashed line). Data is shown for sequential arm patients only.

FIG. 7 illustrates correlation between 4 continuous covariates at baseline. WTKGB is baseline weight (median is 78.3, excluding EFC16295). BH is baseline race (median is 43, excluding EFC16295). BVWF is baseline VWF (median is 112, excluding EFC16295).

FIGS. 8A and 8B are graphs demonstrating population predictions (PRED) (FIG. 8A) and individual predictions (IPRED) (FIG. 8B) versus or observed value of one-stage FVIII activity (DV; data value) using efanesoctocog alfa population PK model [A]. Black line is line of unity. Grey line is loess smoothing line. R²=0.92 for the population predictions in FIG. 8A. R²=0.97 for the individual predictions in FIG. 8B.

FIG. 9 is a set of graphs demonstrating the performance of efanesoctocog alfa population PK model [A] using visual predictive checks (VPC). Open circles show observed data. Solid line shows the model simulated median. Dashed lines show model simulated 5^{th} and 95^{th} percentiles. Shading around each dashed line represents 90% CI around the simulated 5^{th} and 95^{th} percentiles. Shading around each solid line shows the 90% CI around the simulated median.

FIGS. 10A and 10B demonstrate PRED (FIG. 10A) and IPRED (FIG. 10B) versus DV for surgery using efanesoctocog alfa population PK model [A]. The grey line is the line of unity. R² is shown as a black line which is the regression line between observed and predicted.

FIG. 11 is a set of graphs showing the distribution of steady state C_{trough}, Cₘₐₓₛₛ and time to 40 IU/dL FVIII activity levels across populations.

FIG. 12 is a set of graphs showing the distribution of steady state C_{trough}, Cₘₐₓₛₛ and time to 40 IU/dL FVIII activity levels across non-Asian and Asian populations.

FIG. 13 is a graph showing OSC FVIII activity over time for major surgery and major bleeds for patients age 6 and younger. Grey solid line: simulated median for 30 IU/kg dose. Grey line with circles: simulated median for 50 IU/kg dose. Black solid lines: simulated 5th and 95th percentiles. Dashed lines indicate OSC activity of 80 IU/kg and 40 IU/kg.

FIG. 14 is a graph showing simulated FVIII activity for a dose of 50 IU/kg efanesoctocog alfa followed by 30 IU/kg every 3 days until Day 14 in a virtual adult and adolescent population

FIG. 15 is a graph showing simulated OSC FVIII activity over time for all age groups.

### DETAILED DESCRIPTION

With the emergence of extended half-life replacement products, treatment goals are now expanding beyond targeting a low annualized bleed rate (ABR) to include long-term outcomes associated with high sustained plasma FVIII activity levels, such as long-term joint protection. Efanesoctocog alfa circulates independently of endogenous von Willebrand factor (VWF), and provides high sustained FVIII activity (see, e.g., Chhabra, et al. Blood. 2020; 135 (17): 1484-1496 and Konkle et al., N Engl J Med 2020; 383:1018-1027 (referring to efanesoctocog alfa as BIVV001), the entire contents of each of which are incorporated herein by reference for all purposes).

The present disclosure provides, inter alia, methods of treatment and software-based systems for estimating individual subject efanesoctocog alfa PK information for treatment of hemophilia A. For example, methods of estimating individualized subject efanesoctocog alfa PK information using the software-based system are included. In some embodiments, the software-based system applies an efanesoctocog alfa population PK model [A] for estimating dose information for subjects receiving efanesoctocog alfa as FVIII replacement treatment.

The present disclosure provides, inter alia, methods of treatment and software-based systems for estimating or quantifying the risk of bleed with high sustained FVIII activity. In some embodiments, the software-based system can apply an efanesoctocog alfa population pharmacokinetic/pharmacodynamic model for quantifying the risk of bleed with high sustained FVIII activity compared to standard of care.

### Definitions

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some embodiments, the term indicates deviation from the indicated numerical value by ±10%, ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, or ±0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%.

It is understood that wherever aspects are described herein with the language “comprising,” otherwise analogous aspects described in terms of “consisting of” and/or “consisting essentially of” are also provided.

As used herein in the context of hemophilia A, the term “prophylactic treatment” refers to the preemptive administration of a therapy for the treatment of hemophilia A, where such treatment is intended to prevent or reduce the severity of one or more symptoms of hemophilia A, e.g., bleeding episodes, such as one or more spontaneous bleeding episodes, and/or joint damage. To prevent or reduce the severity of such symptoms, e.g., bleeding episodes and the progression of joint disease, hemophilia A patients may receive regular infusions of clotting factor (such as efanesoctocog alfa) as part of a prophylactic treatment regimen.

The term “on-demand treatment” or “episodic treatment” refers to the “as needed” administration of a FVIII replacement therapy (such as efanesoctocog alfa) in response to symptoms of hemophilia A, e.g., a bleeding episode (such as a spontaneous bleeding episode or a traumatic bleeding episode), or before an activity that can cause bleeding. In some embodiments, the on-demand treatment can be given to a subject when bleeding starts, such as after an injury, or when bleeding is expected, such as before surgery. In some embodiments, the on-demand treatment can be given prior to activities that increase the risk of bleeding, such as contact sports. In some embodiments, on-demand treatment can be administered to a subject who is receiving prophylactic treatment, e.g., if supplemental FVIII replacement protein doses are administered to treat a bleeding episode or before strenuous activity. In some embodiments, the on-demand treatment is given as a single dose. In some embodiments, the on-demand treatment is given as a first dose, followed by one or more additional doses. In some embodiments, the on-demand regimen is for perioperative management of bleeding.

In some embodiments, a bleeding episode starts from the first sign of a bleed and ends 72 hours after the last treatment for the bleeding, within which any symptoms of bleeding at the same location, or injections less than or equal to 72 hours apart, is considered the same bleeding episode. See Blanchette V. (2006) Haemophilia 12:124-7. In some embodiments, any injection to treat the bleeding episode, taken more than 72 hours after the preceding one, is considered the first injection to treat a new bleeding episode at the same location. In some embodiments, any bleeding at a different location is considered a separate bleeding episode regardless of time from the last injection.

The methods provided herein can be applied to a subject in need of prophylactic treatment or episodic/on-demand treatment. In some embodiments, the subject in need of prophylactic treatment or episodic/on-demand treatment suffers from hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, and bleeding in the iliopsoas sheath. In some embodiments, the subject is in need of treatment for surgery, including, e.g., surgical prophylaxis or perioperative management. In some embodiments, the surgery is minor surgery or major surgery. Exemplary surgical procedures include tooth extraction, tonsillectomy, inguinal herniotomy, synovectomy, craniotomy, osteosynthesis, trauma surgery, intracranial surgery, intra-abdominal surgery, intrathoracic surgery, joint replacement surgery (e.g., total knee replacement, hip replacement, and the like), heart surgery, and caesarean section.

“Treat” and “treating”, as used herein in the context of hemophilia A include, e.g., the reduction in severity of hemophilia A; the amelioration of one or more symptoms associated with hemophilia A; the provision of beneficial effects to a subject with hemophilia A, without necessarily curing the hemophilia A; and/or the prophylaxis of one or more symptoms associated with hemophilia A.

In some embodiments, treating hemophilia A includes prevention of one or more symptoms of hemophilia A (such as spontaneous bleeding). In some embodiments, treating hemophilia A includes reducing the likelihood of a bleeding episode or reducing the severity of a bleeding episode. In some embodiments, treatment is prophylactic treatment. In some embodiments, treatment is on-demand treatment. In some embodiments, treating comprises the reduction of the frequency of one or more symptoms of hemophilia A, e.g., spontaneous or uncontrollable bleeding episodes.

The term “perioperative management” as used herein means use of efanesoctocog alfa before, concurrently with, or after an operative procedure, e.g., a surgical operation. The use for “perioperative management” of one or more bleeding episode includes surgical prophylaxis before (i.e., preoperative), during (i.e., intraoperative), or after (i.e., postoperative) a surgery to prevent one or more bleeding or bleeding episode or reducing or inhibiting spontaneous and/or uncontrollable bleeding episodes before, during, and after a surgery.

As used herein, a “baseline” plasma FVIII level is the lowest measured plasma FVIII level in a subject prior to administering a dose. In some embodiments, activity above the baseline pre-dosing can be considered residue FVIII activity from prior treatment, and can be decayed with time using the half-life of prior treatment and subtracted from the PK data following efanesoctocog alfa dosing. In some embodiments, the baseline FVIII activity level is the level of FVIII activity in the blood (e.g. as assessed with plasma) of the patient in the absence of treatment for hemophilia A.

The terms “patient” and “subject” are used interchangeably herein and refer to a human. A subject may include, e.g., an individual who has been diagnosed with hemophilia A, and who is susceptible to spontaneous and/or uncontrolled bleeding episodes. Subjects can also include individuals who are in danger of one or more uncontrollable bleeding episodes prior to a certain activity, e.g., a surgery, a sport activity, or any strenuous activity. In some embodiments, the subject has a baseline FVIII activity less than 0.5%, less than 1%, less than 2%, less than 2.5%, less than 3%, or less than 4%. In some embodiments, the subject has severe hemophilia A, defined as <1 IU/dL (<1%) endogenous FVIII activity. In some embodiments, the subject has no coagulation disorder other than hemophilia A.

As used herein, the terms “ELNN polypeptide” and “ELNN” are synonymous, and refer to extended length polypeptides comprising non-naturally occurring, substantially non-repetitive sequences (e.g., polypeptide motifs) that are composed mainly of small hydrophilic amino acids, with the sequence having a low degree or no secondary or tertiary structure under physiologic conditions. Such extended length polypeptides include unstructured hydrophilic polypeptides comprising repeating motifs of 6 natural amino acids (G, A, P, E, S, and/or T). In some embodiments, an ELNN polypeptide comprises multiple motifs of 6 natural amino acids (G, A, P, E, S, T), wherein the motifs are the same or comprise a combination of different motifs. ELNN polypeptides can confer certain desirable pharmacokinetic, physicochemical and pharmaceutical properties when linked to a VWF fragment or a FVIII sequence of the disclosure to create a chimeric polypeptide or protein. Such desirable properties include but are not limited to enhanced pharmacokinetic parameters and solubility characteristics. ELNN polypeptides are known in the art, and non-limiting descriptions relating to and examples of ELNN polypeptides known as XTEN polypeptides are available in Schellenberger et al., (2009) Nat Biotechnol 27 (12): 1186-90; Brandl et al., (2020) Journal of Controlled Release 327:186-197; and Radon et al., (2021) Advanced Functional Materials 31, 2101633 (pages 1-33), the entire contents of each of which are incorporated herein by reference.

As used herein “software-based system” refers to an algorithm or set of algorithms capable of being implemented by a processing device. The software-based system may be embodied in software which includes but is not limited to firmware, resident software, microcode, etc. and may take the form of a computer program product accessible from a computer-usable or computer-readable medium providing program code for use by or in connection with a computer or any instruction execution system. For the purposes of this description, a computer-usable or computer readable medium can be any apparatus that can contain, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk, and an optical disk, including compact disc-read only memory (CD-ROM), compact disc-read/write (CD-R/W) and DVD. Non-limiting examples of software-based systems include network-based systems and web-based systems.

As used herein the term “processing device” refers to a data processing system suitable for storing and/or executing program code to implement the software-based system and may include at least one processor coupled directly or indirectly to memory elements through a system bus. The processor(s), the electronic circuitry that executes instructions that make up the program code, may be instantiated by a microprocessor, microcontroller, multi-core processor, array of processors, or vector processors. The memory elements can include local memory employed during actual execution of the program code, bulk storage, and cache memories which provide temporary storage of at least some program code in order to reduce the number of times code must be retrieved from bulk storage during execution. Input/output or I/O devices (including but not limited to keyboards, displays, pointing devices, touch screens, audio, etc.) can be coupled to the system either directly or through intervening I/O controllers. Network adapters may also be coupled to the system to enable the processing device to become coupled to other processing devices or remote printers or storage devices through intervening private or public networks. Modems, cable modems and Ethernet cards are just a few of the currently available types of network adapters. The processing device may also be a shared data processing system such as a network-based (e.g., web-based) server system, accessible via a network such as the Internet, that is capable of accessing and executing program code to implement the software-based system.

### Description of Efanesoctocog Alfa

Efanesoctocog alfa is described in Chhabra et al. Blood 2020; 135 (17): 1484-1496, Konkle et al., N Engl J Med 2020; 383:1018-1027, and the International Nonproprietary Names for Pharmaceutical Substances (INN) WHO Drug Information, 2019, Vol. 33, No. 4, p. 828-30, the entire contents of each of which are hereby incorporated by reference in their entireties. Efanesoctocog alfa temporarily replaces the missing FVIII needed for effective hemostasis in patients with a deficiency of FVIII. Efanesoctocog alfa comprises a first polypeptide comprising the amino acid sequence of SEQ ID NO: 3 covalently bound to a second polypeptide comprising the amino acid sequence of SEQ ID NO: 6, wherein the first polypeptide and the second polypeptide are covalently bound to each other via disulfide bonds. Efanesoctocog alfa may be produced, e.g., by recombinant DNA technology in a human embryonic kidney (HEK) cell line. For example, the cell line can express an rFVIIIFc-ELNN polypeptide (SEQ ID NO: 1), an rVWF-ELNN-Fc polypeptide (SEQ ID NO: 4), and a soluble PACE enzyme. Non-limiting examples of nucleotide sequences encoding the rFVIIIFc-ELNN polypeptide (SEQ ID NO: 2) and the rVWF-ELNN-Fc polypeptide polypeptide (SEQ ID NO: 5) can be found in Table 7, below. Amino acid sequences for the rFVIIIFc-ELNN polypeptide without a signal peptide (SEQ ID NO: 3) and rVWF-ELNN-Fc polypeptide polypeptide without a signal peptide or D1D2 portion of VWF (SEQ ID NO: 6) can be found in Table 7, below.

In some embodiments for subjects receiving prophylactic treatment with efanesoctocog alfa, the methods disclosed herein can be used to determine individualized subject information (e.g., the subject's plasma FVIII levels at a particular time point or a set of timepoints). Based on this individual subject information, the dose and/or dose interval of efanesoctocog alfa can be adjusted to achieve individual treatment goals, such as a minimum plasma FVIII level (e.g., trough).

In some embodiments, for subjects receiving on-demand treatment with efanesoctocog alfa, if the subject's bleeding is not controlled or is unsatisfactory after efanesoctocog alfa administration at an initial recommended dose and dose interval, the methods disclosed herein can be used to determine individual subject dose information. Based on this individual subject dose information, the dose and/or dose interval of efanesoctocog alfa can be adjusted to achieve improved bleed control.

In some embodiments, the methods disclosed herein can be used to estimate a minimum FVIII level between doses for a subject.

Subject's plasma can be monitored for FVIII activity levels, e.g., the one-stage clotting assay, to confirm adequate FVIII levels have been achieved and maintained, when clinically indicated. FVIII activity can be measured by any known methods in the art. A number of tests are available to assess the function of the coagulation system: activated partial thromboplastin time (aPTT) test, chromogenic assay, ROTEM assay, prothrombin time (PT) test (also used to determine INR), fibrinogen testing (often by the Clauss method), platelet count, platelet function testing (often by PFA-100), TCT, bleeding time, mixing test (whether an abnormality corrects if the subject's plasma is mixed with normal plasma), coagulation factor assays, antiphospholipid antibodies, D-dimer, genetic tests (e.g., factor V Leiden, prothrombin mutation G20210A), dilute Russell's viper venom time (dRVVT), miscellaneous platelet function tests, thromboelastography (TEG or Sonoclot), thromboelastometry (TEM®, e.g., ROTEM®), or euglobulin lysis time (ELT).

The aPTT test is a performance indicator measuring the efficacy of both the “intrinsic” (also referred to the contact activation pathway) and the common coagulation pathways. This test is commonly used to measure clotting activity of commercially available recombinant clotting factors, e.g., FVIII. It is typically used in conjunction with prothrombin time (PT), which measures the extrinsic pathway. (See, e.g., Kamal et al., Mayo Clin Proc., 82 (7): 864-873 (2007)). In some embodiments, aPTT is tested using an assay where FVIII activity is measured using the Dade® Actin® FSL Activated PTT Reagent (Siemens Health Care Diagnostics) on a BCS® XP analyzer (Siemens Healthcare Diagnostics).

In some embodiments, the aPTT assay may also be used for assessing the potency of a chimeric polypeptide prior to administration to a subject. (Hubbard A R, et al. J Thromb Haemost 11:988-9 (2013)). In some embodiments, the aPTT assay may further be used in conjunction with any of the assays described herein, either prior to administration or following administration to a subject.

In some embodiments, a model provided herein provides FVIII activity calculations and information that correspond to activity as measured by aPTT test. For example, efanesoctocog alfa popPK model [A] provides FVIII activity calculations and information that correspond to activity as measured by aPTT test.

ROTEM analysis provides information on the whole kinetics of hemostasis: clotting time, clot formation, clot stability and lysis. The different parameters in thromboelastometry are dependent on the activity of the plasmatic coagulation system, platelet function, fibrinolysis, or many factors which influence these interactions. This assay can provide a complete view of secondary hemostasis.

The chromogenic assay mechanism is based on the principles of the blood coagulation cascade, where activated FVIII accelerates the conversion of Factor X into Factor Xa in the presence of activated Factor IX, phospholipids and calcium ions. The Factor Xa activity is assessed by hydrolysis of a p-nitroanilide (pNA) substrate specific to Factor Xa. The initial rate of release of p-nitroaniline measured at 405 nM is directly proportional to the Factor Xa activity and thus to the FVIII activity in the sample. In some embodiments, the chromogenic assay is the BIOPHEN FVIII: C assay (Hyphen Biomed, Neurville sur Oise, France). In some embodiments, the chimeric polypeptide comprising a FVIII polypeptide has FVIII activity comparable to a chimeric polypeptide comprising mature FVIII polypeptide or a BDD FVIII polypeptide (e.g., RECOMBINATE®, KOGENATE FS®, HELIXATE FS®, XYNTHA/REFACTO AB®, HEMOFIL-M®, MONARCM®, MONOCLATE-PR, HUMATE-P®, ALPHANATE®, KOATE-DVI®, AFSTYLA®, AND HYATE: C®).

In some embodiments, a chromogenic assay may also be used for assessing the potency of a chimeric polypeptide prior to administration to a subject. (Hubbard A R, et al. J Thromb Haemost 11:988-9 (2013)). The chromogenic assay may further be used in conjunction with any of the assays described herein, either prior to administration or following administration to a subject.

### Subject Dosing Information

For most currently available FVIII replacement therapies, the required FVIII dose for each subject is calculated using the following formula:

$$Number of factor VIII units required ( IU ) = Body Weight ( kg ) \times Desired FVIII Increase ( IU / dL or \% of normal ) \times 0.5 ( IU / kg per IU / dL ) ) \qquad [ B ]$$

This calculation provides a general estimation of the dosing requirements of a subject based on body weight as a subject specific variable and the desired FVIII activity level increase.

Disclosed herein is a model [A] for determining or estimating dosing information for hemophilia A subjects receiving FVIII replacement therapy with efanesoctocog alfa. The efanesoctocog alfa popPK model [A] is represented as follows:

$$CL = TVCL * {( WT / 78.3 )}^{0.677} * {( 0.896 )}^{ASIAN} * {e}^{η 1} \\ V = TVV * {( WT / 78.3 )}^{0.791} * {e}^{η2} \\ k = CL / V \\ \frac{{dA}_{1}}{dt} = Rate - {kA}_{1} \\ C = {A}_{1} / V \qquad [ A ]$$

Model [A] Abbreviations are: CL, clearance from central compartment; TVCL, estimate of typical clearance; WT, bodyweight in kg; ASIAN, indicator for Asian race (0 for Non-Asians and 1 for Asians); η₁, variability on CL; V, volume of central compartment; TVV, estimate of typical volume; η₂, variability on V; k, elimination rate from central compartment; Rate, rate of infusion; A₁, amount of one-stage (OS) FVIII activity in central compartment;
and C, one-stage (OS) FVIII activity in central compartment.

Investigation into covariates that affect FVIII activity has historically focused on VWF concentration. However, VWF concentration was not found to be a covariate in the efanesoctocog alfa popPK data and model disclosed herein. Without being bound by any scientific theory, it is noted that efanesoctocog alfa clearance is independent of endogenous VWF. Hematocrit level was also found to not be a covariate.

For the efanesoctocog alfa popPK model disclosed herein, clearance from central compartment (CL) and volume of central compartment (V) were found to depend on bodyweight. Asian race was also identified as a covariate on CL.

In some embodiments, race is a subject's self-reported race. For example, a subject may self-report from a set of possible options that include the option “Asian”. In some embodiments, if a subject decides to not report a race, the subject is considered as other than Asian for the purposes of the model. In some embodiments, when self-reported race information is not or cannot be collected, the subject is considered as other than Asian for the purposes of the model. In some embodiments, a subject who self-reports as Asian self-identifies as being of East Asian descent or ancestry. In some embodiments, a subject who self-reports as Asian self-identifies as being of Southeast Asian descent or ancestry. In some embodiments, a subject who self-reports as being Asian self-identifies as being of Central Asian descent or ancestry. In some embodiments, a subject who self-reports as being Asian self-identifies as being of North Asian descent or ancestry. In some embodiments, a subject who self-reports as being Asian self-identifies as being of West Asian descent or ancestry. In some embodiments, the subject self-identifies as being of East Asian descent or ancestry. In some embodiments, the subject self-identifies as being of South Asian descent or ancestry. In some embodiments, the subject self-identifies as being of Central Asian descent or ancestry. In some embodiments, the subject self-identifies as being of Southeast Asian descent or ancestry. In some embodiments, the subject self-identifies as being of West Asian descent or ancestry.

Some embodiments comprise administering a dose of efanesoctocog alfa to a human subject in need thereof at a dosing interval, wherein the dose and/or the dosing interval is identified using the subject's body weight and/or self-reported race, but not the subject's VWF or hematocrit levels. The present disclosure provides methods of administering a dose of efanesoctocog alfa to a human subject in need thereof at a dosing interval, wherein the dose and/or the dosing interval is identified by applying the efanesoctocog alfa model [A] disclosed herein.

The efanesoctocog alfa model [A] disclosed herein can be used to determine individual subject dosing information in order to evaluate and/or confirm subject treatment goals. This individual subject dosing information can be used to determine dose and/or dosing interval of efanesoctocog alfa in future treatment. Treatment goals may include, e.g., higher plasma FVIII levels over long periods of time.

In some embodiments, the therapeutically effective dose of efanesoctocog alfa is about 50 IU/kg. In some embodiments, the subject is administered a dose of about 50 IU/kg once-weekly. In some embodiments, the subject is administered a dose of about 50 IU/kg once every about 7 days. In some embodiments, the subject is administered an initial dose of about 50 IU/kg, followed by either 50 IU/kg or 30 IU/kg every 2-3 days as needed.

In some embodiments, the methods disclosed herein are applied to determine a subject's individualized interval prophylaxis. The term “individualized interval prophylaxis” as used herein means use of efanesoctocog alfa for an individualized dose and/or dosing interval or frequency to prevent or inhibit occurrence of one or more spontaneous and/or uncontrollable bleeding or bleeding episodes or to reduce the frequency of one or more spontaneous and/or uncontrollable bleeding or bleeding episodes.

In some embodiments, subject treatment goals include achieving high FVIII plasma activity levels and/or high trough levels. As used herein, a “trough level” in a hemophilia subject is the measurement of the lowest concentration reached by a factor therapy, e.g., efanesoctocog alfa therapy, before the next dose is administered. The methods disclosed herein can be used to determine subject dosing information in order to achieve specific FVIII plasma activity levels and/or trough levels. Administration of efanesoctocog alfa has been shown to successfully achieve high FVIII plasma activity levels and/or high trough levels in hemophilia A subjects.

In some embodiments, administration of efanesoctocog alfa results in a level of FVIII activity of 40% or greater in the subject for about 1, 2, 3, or 4 days. In some embodiments, administration of efanesoctocog alfa results a level of FVIII activity of greater in the subject for about 1, 2, 3, or 4 days. In some embodiments, administration of efanesoctocog alfa results in a level of FVIII activity of at least 40% in the subject for at least three days. In some embodiments, administration of efanesoctocog alfa results in a level of FVIII activity of at least 50% in the subject for about 4 days. In some embodiments, the efanesoctocog alfa model [A] is used to determine individual subject dosing information in order to achieve a FVIII activity level of at least 40% in the subject for about 1, 2, 3 or 4 days.

### Method, System, and Storage Medium for Estimating Subject Individualized Dosing Information, Subject Individualized PK Information, and Subject Median PK Information

Included herein is a method of estimating (e.g., calculating, determining, or providing) individualized efanesoctocog alfa dosing information for an individual subject, the method comprising: (a) receiving subject information and/or desired treatment outcome information by an application program programmed to operate with an efanesoctocog alfa popPK model (e.g., efanesoctocog alfa popPK model [A]), b) calculating individualized efanesoctocog alfa dosing information using the efanesoctocog alfa popPK model and the received information, and c) outputting the calculated individualized efanesoctocog alfa dosing information of (b). Further disclosed is the method as described herein, further comprising selecting a dosing regimen based on the output individualized dosing information of (c) and administering the efanesoctocog alfa to the subject according to the selected dosing regimen. One or more of the above steps may be performed using one or more of a software-based system, a network-based system, a computing system, or various combinations of the aforementioned-systems. For example, the exemplary network-based system can be used for obtaining an estimated subject individualized dosing information, subject individualized PK information, and subject median PK information.

In some embodiments (a) further comprises receiving, by the software-based system, subject information.

In some embodiments the subject information comprises age, self-reported race, and/or body weight. Additional subject information can further include diagnostic (baseline) FVIII level, PK determinations, time of PK sampling, dosing history if PK samples were taken from multiple doses, actual dose, FVIII activity level, etc.

In some embodiments, output information comprises, e.g., PK curve, PK parameter such as incremental recovery (Cmax/dose), mean residence time, terminal t½, clearance, Vss, AUC/dose, doses and associated troughs, and intervals and associated troughs.

For example, for assessing individualized subject PK, the system can recommend that the user input 2-3 optimized PK sampling time points. In this case, system output can include PK curve and one or more selected PK parameters.

As additional examples, to select an individualized dosing regimen using the output individual PK parameters discussed in the preceding paragraph, (i) the dose selected for acute treatment can be based on user input of the desired rise in plasma FVIII activity level following the dose, (ii) the dose selected for prophylaxis can be based on user input of the desired dosing interval, or (iii) the selected interval for prophylaxis can be based on user input for the desired dose. In the second case, system output can be a table of doses and associated troughs, e.g., x IU/kg, 10% trough, y IU/kg, 20% trough, etc. In the third case, system output can be a table of intervals and associated troughs, e.g., x days, 10% trough, y IU/kg, 20% trough, etc.

In some embodiments, the user may wish to use the system without inputting any individualized PK data. In some embodiments, the dosing output would be based on the population mean or median rather than being individualized for the particular subject. In some embodiments, the user inputs, e.g., body weight and/or self-reported race, and (i) the desired rise in plasma FVIII activity level following the dose, (ii) the desired dose interval for prophylaxis, or (iii) the desired dose for prophylaxis. In the first case, the system can output the dose. In the second case, the system can output the dose and associated trough. In the third case, the system can output the interval and associated trough.

The system may be compliant with patient privacy laws. In some embodiments, the system is encrypted, e.g., with SSL. In some embodiments, input subject information is made anonymous.

In some embodiments, the system includes a user help function.

In some embodiments, the method can be carried out by, e.g., a subject, a physician, a nurse, or another healthcare practitioner. In some embodiments, the method is carried out by the subject.

Some embodiments include a computer readable storage medium having instructions stored thereon that, when executed by a processor, cause the processor to perform one or more steps of the above methods.

Some embodiments include a system comprising a processor and a memory, the memory having instructions stored thereon that, when executed by the processor, cause the processor to perform any of the above methods.

The user of the system or computer readable storage medium can be, e.g., a subject or a caregiver, or a physician, a nurse, or other healthcare practitioner.

In some embodiments, the subject information entered into the system includes body weight. In some embodiments, the subject information entered into the system is self-reported race. In some embodiments, the desired treatment outcome information is desired rise in plasma FVIII activity level following dosing and the output information is dose for acute treatment. In some embodiments, the desired treatment outcome information is desired dosing interval and the output information is dose for prophylaxis. In some embodiments, the desired treatment outcome information is desired dose and the output information is interval for prophylaxis.

In some embodiments, the individual efanesoctocog alfa PK information includes 2-3 PK sampling time points. In some embodiments, the individual efanesoctocog alfa PK information includes one or more of subject body weight, diagnostic (baseline) factor level, dosing history if PK samples were taken from multiple doses, actual dose, actual time of PK sampling, factor activity level, subject body weight, and/or subject self-reported race.

In some embodiments the output individualized subject PK includes a PK curve or a PK parameter selected from incremental recovery (Cmax/Dose), mean residence time, terminal t½, clearance, Vss and AUC/Dose. In some embodiments, the desired treatment outcome information based on the individual subject's PK is desired rise in plasma FVIII activity level following dosing and the output information is dose for acute treatment.

In some embodiments, the methods disclosed herein include an electronic device. An electronic device can include, but is not limited to, a device having a processor and memory for executing and storing instructions. The electronic device may also include a display and one or more computer input devices such as a keyboard, a mouse, a pad, a touch screen, a microphone, and/or a joystick. In some embodiments, the electronic device is a general-purpose computing and data communication device such as digital pen, a smart phone, a smart watch, a tablet computer, a personal digital assistant, a handheld computer, a laptop computer, a point-of-sale transaction device, a scanner, a camera, and a fax machine. The electronic device may also have multiple processors and multiple shared or separate memory components. For example, the electronic device may be a clustered computing environment or server farm.

Alternatively, the electronic device can be a specialized data collection, computing and communications device such as, for example, a point-of-care (POC) device capable of receiving subject demographic information including age, vital signs including body weight, and/or blood characterizing values including self-reported race. The blood characterizing values may be received by the electronic device via a data communications channel, manual entry, and/or by diagnostic processes performed by the electronic device. Diagnostic processes performed on subject blood samples within the device may include ultrasound measurements, impedance measurements, conductivity measurements, and/or optical measurements. The electronic device may be further configured to receive, detect, record and/or communicate additional subject information including diagnostic (baseline) FVIII level, PK determinations, time of PK sampling, dosing history if PK samples were taken from multiple doses, actual dose, FVIII activity level. The electronic device communicates with one or more network-based (e.g., web-based) application programs over one or more networks, such as the Internet. Similar to the electronic device, the network-based (e.g., web-based) application program can be implemented using a general-purpose computer, a server, or other device capable of serving data to the electronic device. The electronic device can receive individualized subject efanesoctocog alfa PK information from a network-based (e.g., web-based) server and program. In some embodiments, the electronic device can assist in selecting a dosing regimen based on the output calculated subject PK information.

The methods and systems described herein may be implemented in or via a mobile device. Mobile devices include navigation devices, mobile phones, smart phones, smart watches, tablets, mobile personal digital information processing terminals, laptops, palmtops, netbooks, pagers, electronic book terminals, music players, and the like. These devices, apart from other components, may comprise a storage medium such as flash memory, buffers, RAM, ROM and one or more computing devices. A computing device associated with the mobile device may be adapted to execute program code, methods, and instructions stored thereon. As another example, a mobile device may be configured to execute instructions in cooperation with other devices. The mobile device may communicate with a base station that is connected to the server and configured to execute the program code. Mobile devices can also communicate over peer-to-peer networks, mesh networks, or other communication networks. The program code may be stored in a storage medium associated with the server and executed by a computing device embedded in the server. The base station may comprise a computing device and a storage medium. The storage medium may store program code and instructions that are executed by a computing device associated with the base station. In some embodiments, the methods and systems described herein are directed to a kit for collecting subject information. Although different embodiments of the kit may include different components, an exemplary kit includes a diagnostic device such as a processing element and/or a calculation element for acquiring information from the subject, and a transmission element that transmits the subject information to a computer device through a wired or wireless connection. The transmitting element in the kit may be configured to transmit subject information in real time when the device is in use, or the diagnostic information may be transmitted with receipt of instructions from a user or provider. Any of the components of the kit, such as the body, can be configured as a hands-free unit during use or as a handheld unit during use.

### Exemplary Computing Environments for the Disclosed Methods and Systems

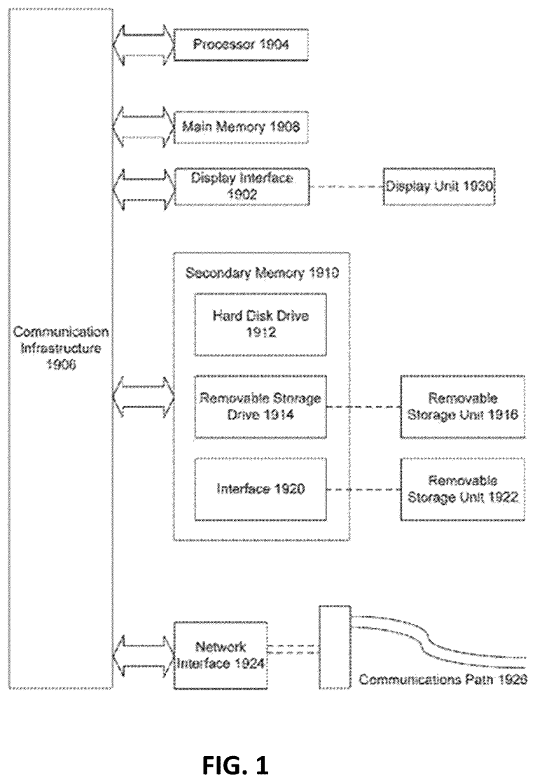

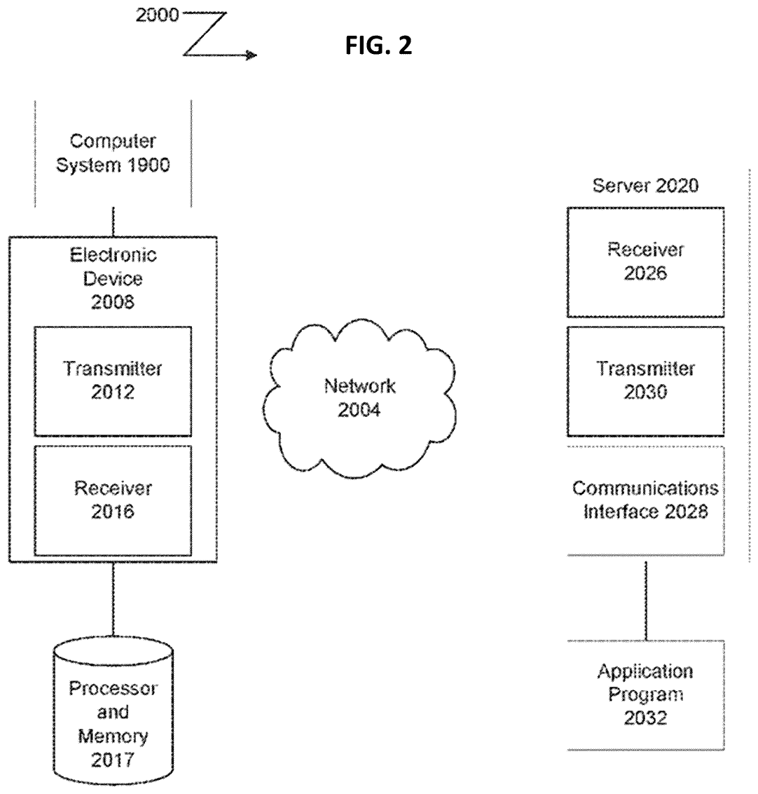

Various modeling techniques, dosage calculations, and estimations described herein can be implemented by software, firmware, hardware, or a combination thereof. FIG. 1 illustrates an example computer system 1900 in which the embodiments, or portions thereof, can be implemented as computer-readable code. In another embodiment, for efanesoctocog alfa, the modeling disclosed in the Examples herein can be implemented in system 1900.

Computer system 1900 includes one or more processors, such as processor 1904. Processor 1904 is connected to a communication infrastructure 1906 (for example, a bus or network).

Computer system 1900 also includes a main memory 1908, preferably random access memory (RAM), and may also include a secondary memory 1910. In accordance with implementations, user interface data may be stored, for example and without limitation, in main memory 1908. Main memory 1908 may include, for example, cache, and/or static and/or dynamic RAM. Secondary memory 1910 may include, for example, a hard disk drive and/or a removable storage drive. Removable storage drive 1914 may include a floppy disk drive, a magnetic tape drive, an optical disk drive, a flash memory, or the like. The removable storage drive 1914 reads from and/or writes to removable storage unit 1916 in a well-known manner. Removable storage unit 1916 may include a floppy disk, magnetic tape, optical disk, etc. which is read by and written to by removable storage drive 1914. As will be appreciated by persons skilled in the relevant art(s), removable storage unit 1916 includes a computer readable storage medium having stored therein computer software and/or data.

Computer system 1900 may also include a display interface 1902. Display interface 1902 may be adapted to communicate with display unit 1930. Display unit 1930 may include a computer monitor or similar means for displaying graphics, text, and other data received from main memory 1908 via communication infrastructure 1906. In alternative implementations, secondary memory 1910 may include other similar means for allowing computer programs or other instructions to be loaded into computer system 1900. Such means may include, for example, a removable storage unit 1922 and an interface 1920. Examples of such means may include a program cartridge and cartridge interface, a removable memory chip (such as an EPROM, or PROM) and associated socket, and other removable storage units 1922 and interfaces 1920 which allow software and data to be transferred from the removable storage unit 1922 to computer system 1900.

Computer system 1900 may also include a communications interface 1924. Communications interface 1924 allows software and data to be transferred between computer system 1900 and external devices. Communications interface 1924 may include a modem, a network interface (such as an Ethernet card or WiFi), a communications port, a PCMCIA slot and card, or the like. Software and data transferred via communications interface 1924 are in the form of signals which may be electronic, electromagnetic, optical, or other signals capable of being received by communications interface 1924. These signals are provided to communications interface 1924 via a communications path 1926. Communications path 1926 carries signals and may be implemented using wire or cable, fiber optics, a phone line, a cellular phone link, WiFi, Bluetooth, an RF link or other communications channels.

In this document, the term “computer readable storage medium” is used to generally refer to non-transitory storage media such as removable storage unit 1916, removable storage unit 1922, and a hard disk installed in hard disk drive 1912. Computer readable storage medium can also refer to one or more memories, such as main memory 1908 and secondary memory 1910, which can be memory semiconductors (e.g., DRAMs, etc.). These computer program products are means for providing software to computer system 1900.

Computer programs (also called computer control logic) are stored in main memory 1908 and/or secondary memory 1910. Computer programs may also be received via communications interface 1924 and stored on main memory 1908 and/or secondary memory 1910. Such computer programs, when executed, enable computer system 1900 to implement embodiments as discussed herein. In particular, the computer programs, when executed, enable processor 1904 to implement processes of the present disclosure, such as certain methods discussed above. Accordingly, such computer programs represent controllers of the computer system 1900. Where embodiments use software, the software may be stored in a computer program product and loaded into computer system 1900 using removable storage drive 1914, interface 1920, or hard drive 1912.

Embodiments may be directed to computer program products comprising software stored on any computer readable medium. Such software, when executed in one or more processing devices, causes a processing device to operate as described herein. Embodiments may employ any computer useable or readable medium. Examples of computer readable storage media include, but are not limited to, non-transitory primary storage devices (e.g., any type of random access memory), and non-transitory secondary storage devices (e.g., hard drives, floppy disks, CD ROMS, ZIP disks, tapes, magnetic storage devices, and optical storage devices, MEMS, nano-technological storage device, etc.). Other computer readable media include communication mediums (e.g., wired and wireless communications networks, local area networks, wide area networks, intranets, etc.).

Non-limiting examples of software-based systems include network-based systems and web-based systems.

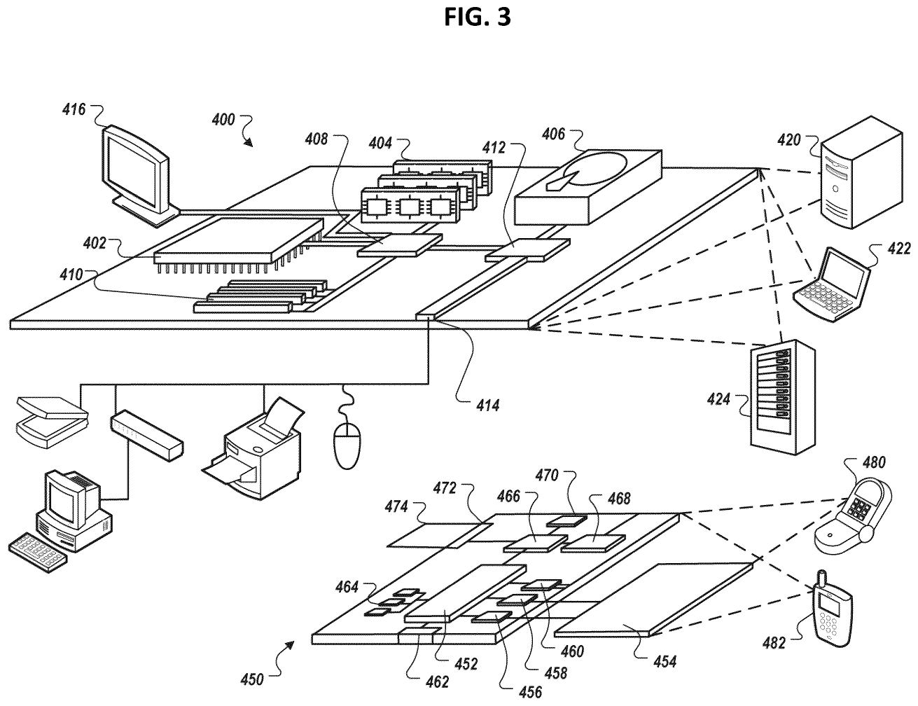

FIG. 3 shows an example of a computing device 400 and an example of a mobile computing device that can be used to implement the techniques described here. The computing device 400 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, and other similar computing devices. The components shown here, their connections and relationships, and their functions, are meant to be exemplary only, and are not meant to limit implementations of the inventions described and/or claimed in this document.

The computing device 400 includes a processor 402, a memory 404, a storage device 406, a high-speed interface 408 connecting to the memory 404 and multiple high-speed expansion ports 410, and a low-speed interface 412 connecting to a low-speed expansion port 414 and the storage device 406. Each of the processor 402, the memory 404, the storage device 406, the high-speed interface 408, the high-speed expansion ports 410, and the low-speed interface 412, are interconnected using various busses, and can be mounted on a common motherboard or in other manners as appropriate. The processor 402 can process instructions for execution within the computing device 400, including instructions stored in the memory 404 or on the storage device 406 to display graphical information for a GUI on an external input/output device, such as a display 416 coupled to the high-speed interface 408. In other implementations, multiple processors and/or multiple buses can be used, as appropriate, along with multiple memories and types of memory. Also, multiple computing devices can be connected, with each device providing portions of the necessary operations (e.g., as a server bank, a group of blade servers, or a multi-processor system).

The memory 404 stores information within the computing device 400. In some implementations, the memory 404 is a volatile memory unit or units. In some implementations, the memory 404 is a non-volatile memory unit or units. The memory 404 can also be another form of computer-readable medium, such as a magnetic or optical disk.

The storage device 406 is capable of providing mass storage for the computing device 400. In some implementations, the storage device 406 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The computer program product can also be tangibly embodied in a computer- or machine-readable medium, such as the memory 404, the storage device 406, or memory on the processor 402.

The high-speed interface 408 manages bandwidth-intensive operations for the computing device 400, while the low-speed interface 412 manages lower bandwidth-intensive operations. Such allocation of functions is exemplary only. In some implementations, the high-speed interface 408 is coupled to the memory 404, the display 416 (e.g., through a graphics processor or accelerator), and to the high-speed expansion ports 410, which can accept various expansion cards (not shown). In the implementation, the low-speed interface 412 is coupled to the storage device 406 and the low-speed expansion port 414. The low-speed expansion port 414, which can include various communication ports (e.g., USB, Bluetooth, Ethernet, wireless Ethernet) can be coupled to one or more input/output devices, such as a keyboard, a pointing device, a scanner, or a networking device such as a switch or router, e.g., through a network adapter.

The computing device 400 can be implemented in a number of different forms, as shown in the figure. For example, it can be implemented as a standard server 420, or multiple times in a group of such servers. In addition, it can be implemented in a personal computer such as a laptop computer 422. It can also be implemented as part of a rack server system 424. Alternatively, components from the computing device 400 can be combined with other components in a mobile device (not shown), such as a mobile computing device 450. Each of such devices can contain one or more of the computing device 400 and the mobile computing device 450, and an entire system can be made up of multiple computing devices communicating with each other.

The mobile computing device 450 includes a processor 452, a memory 464, an input/output device such as a display 454, a communication interface 466, and a transceiver 468, among other components. The mobile computing device 450 can also be provided with a storage device, such as a micro-drive or other device, to provide additional storage. Each of the processor 452, the memory 464, the display 454, the communication interface 466, and the transceiver 468, are interconnected using various buses, and several of the components can be mounted on a common motherboard or in other manners as appropriate.

The processor 452 can execute instructions within the mobile computing device 450, including instructions stored in the memory 464. The processor 452 can be implemented as a chipset of chips that include separate and multiple analog and digital processors. The processor 452 can provide, for example, for coordination of the other components of the mobile computing device 450, such as control of user interfaces, applications run by the mobile computing device 450, and wireless communication by the mobile computing device 450.

The processor 452 can communicate with a user through a control interface 458 and a display interface 456 coupled to the display 454. The display 454 can be, for example, a TFT (Thin-Film-Transistor Liquid Crystal Display) display or an OLED (Organic Light Emitting Diode) display, or other appropriate display technology. The display interface 456 can comprise appropriate circuitry for driving the display 454 to present graphical and other information to a user. The control interface 458 can receive commands from a user and convert them for submission to the processor 452. In addition, an external interface 462 can provide communication with the processor 452, so as to enable near area communication of the mobile computing device 450 with other devices. The external interface 462 can provide, for example, for wired communication in some implementations, or for wireless communication in other implementations, and multiple interfaces can also be used.

The memory 464 stores information within the mobile computing device 450. The memory 464 can be implemented as one or more of a computer-readable medium or media, a volatile memory unit or units, or a non-volatile memory unit or units. An expansion memory 474 can also be provided and connected to the mobile computing device 450 through an expansion interface 472, which can include, for example, a SIMM (Single In Line Memory Module) card interface. The expansion memory 474 can provide extra storage space for the mobile computing device 450, or can also store applications or other information for the mobile computing device 450. Specifically, the expansion memory 474 can include instructions to carry out or supplement the processes described above, and can include secure information also. Thus, for example, the expansion memory 474 can be provide as a security module for the mobile computing device 450, and can be programmed with instructions that permit secure use of the mobile computing device 450. In addition, secure applications can be provided via the SIMM cards, along with additional information, such as placing identifying information on the SIMM card in a non-hackable manner.

The memory can include, for example, flash memory and/or NVRAM memory (non-volatile random access memory), as discussed below. In some implementations, a computer program product is tangibly embodied in an information carrier. The computer program product contains instructions that, when executed, perform one or more methods, such as those described above. The computer program product can be a computer- or machine-readable medium, such as the memory 464, the expansion memory 474, or memory on the processor 452. In some implementations, the computer program product can be received in a propagated signal, for example, over the transceiver 468 or the external interface 462.

The mobile computing device 450 can communicate wirelessly through the communication interface 466, which can include digital signal processing circuitry where necessary. The communication interface 466 can provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication can occur, for example, through the transceiver 468 using a radio-frequency. In addition, short-range communication can occur, such as using a Bluetooth, WiFi, or other such transceiver (not shown). In addition, a GPS (Global Positioning System) receiver module 470 can provide additional navigation- and location-related wireless data to the mobile computing device 450, which can be used as appropriate by applications running on the mobile computing device 450.

The mobile computing device 450 can also communicate audibly using an audio codec 460, which can receive spoken information from a user and convert it to usable digital information. The audio codec 460 can likewise generate audible sound for a user, such as through a speaker, e.g., in a handset of the mobile computing device 450. Such sound can include sound from voice telephone calls, can include recorded sound (e.g., voice messages, music files, etc.) and can also include sound generated by applications operating on the mobile computing device 450.

The mobile computing device 450 can be implemented in a number of different forms, as shown in the figure. For example, it can be implemented as a cellular telephone 480. It can also be implemented as part of a smart-phone 482, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

Having now described the present disclosure in detail, the same will be more clearly understood by reference to the following examples, which are included herewith for purposes of illustration only and are not intended to be limiting of the present disclosure. All patents and publications referred to herein are expressly incorporated by reference.

### EXAMPLES

### Example 1. Population Pharmacokinetic (PopPK) Model to Characterize Efanesoctocog Alfa Factor VIII (FVIII) Activity Levels in Patients with Severe Hemophilia A

Once-weekly efanesoctocog alfa provides high sustained FVIII activity in the normal to near-normal range for most of the week and demonstrated superior bleed protection compared with prior FVIII prophylaxis. FVIII activity data have been collected from 5 clinical studies (Phase 1/2a single- and repeat-dose studies [NCT03205163 and EudraCT 2018-001535-51, respectively] in adults, and Phase 3 studies in adults and adolescents ≥12 years of age [XTEND-1, NCT04161495] and children ≥1 year to <12 years [XTEND-Kids, NCT04759131], and Phase 3 long-term extension study [XTEND-ed, NCT04644575]). A popPK model was developed to characterize FVIII activity after efanesoctocog alfa dosing, identify intrinsic and extrinsic factors affecting pharmacokinetics (PK), and assess PK variability.

FVIII activity levels used to develop the popPK model were measured by the one-stage clotting assay from 3054 blood samples from 199 adults and adolescents, and 61 children who received efanesoctocog alfa in the aforementioned studies. Body weight and VWF level ranged from 12.5 kg-133 kg and 40 IU/dL-339 IU/dL, respectively. A one-compartment model with linear elimination was used to characterize FVIII activity with an estimated allometric body weight effect on clearance (CL) and volume of central compartment (V) to account for the dependence of CL and V on body size. The efanesoctocog alfa popPK model is shown as equation [A], above.

Baseline VWF, baseline race, race (White and Asian), baseline hematocrit, hepatitis C virus and human immunodeficiency virus status, and blood types (A, B, O) were tested for statistical significance in the covariate analysis. Baseline descriptive statistics of the continuous covariates for the subjects in the final dataset are shown in Table 1. The final popPK model was used to simulate various dose regimens in a virtual population of adult and adolescent patients generated using baseline body weight distribution from the Phase 1/2a studies and XTEND-1.

TABLE 1
Baseline descriptive statistics of the continuous
covariates for the subjects in the final dataset

| | | All Studies |
|---|---|---|
| Covariate | Descriptive statistic | (N = 260) |
| Bodyweight | Mean (SD) | 67.6 (28.5) |
| (kg) | Median (5th-95th percentile) | 72 (17.5-113.0) |
| Age | Mean (SD) | 29.0 (17.9) |
| (year) | Median (5th-95th percentile) | 30.0 (4.0-60.0) |
| Hematocrit | Mean (SD) | 41.7 (4.47) |
| (%) | Median (5th-95th percentile) | 42.0 (34.0-48.0) |
| VWF:RCo | Mean (SD) | 112 (47.2) |
| (IU/dL) | Median (5th-95th percentile) | 100 (53.0-188.0) |

In Table 1, abbreviations are SD, standard deviation; VWF, von Willebrand factor; VWF:RCo, VWF ristocetin cofactor.

### Results:

Table 2 shows PopPK model estimates of Cₘₐₓₛₛ and C_{trough} parameters at Week 26 of efanesoctocog alfa prophylaxis for the adult and adolescent populations, as well as the Cₘₐₓₛₛ and C_{trough} (observed or non-compartmental analysis) at Week 26 from the Phase 3 clinical trials. As shown from the comparisons in Table 2, the PK parameter estimates from the PopPK model are consistent with clinical trial data.

TABLE 2
PopPK parameter estimates at Week 26 of efanesoctocog alfa
prophylaxis for the adult and adolescent population

| | |
|---|---|
| Cₘₐₓₛₛ | C_{troughss} |

Post-hoc PK parameters

| | | |
|---|---|---|
| N | 17 | 17 |
| Mean (SD) | 155 | 15.8 |
| | (27.4) | (5.57) |

Non-compartmental analysis or observed PK parameters

| | | |
|---|---|---|
| N | 17 | 16 |
| Mean (SD) | 154 | 15.2 |
| | (29.7) | (7.31) |

In Table 2, abbreviations are Cmax, maximum concentration; Ctrough, trough concentration; PopPK, population pharmacokinetic; SD, standard deviation; ss, steady-state.

TABLE 3
Parameter estimates of the final popPK
model for efanesoctocog alfa

| Parameter | Estimate | RSE (%) |
|---|---|---|
| Typical value of CL (⊖₁, dL/h) | 0.433 | 1.43 |
| Typical value of V (⊖₂, dL) | 30.2 | 1.04 |
| Power of coefficient of bodyweight on CL | 0.677 | 3.91 |
| Power of coefficient of bodyweight on V | 0.791 | 3.15 |
| Proportional coefficient of Asian Race on CL | 0.896 | 2.56 |

| | Inter-individual variability | | |
|---|---|---|---|
| Parameter | Estimate (% CV) | RSE (%) | Shrinkage (%) |
| CL | 0.0354 | 14.5 | 6.07 |
| | (19.0) | | |
| V | 0.0209 | 14.5 | 12.6 |
| | (14.6) | | |
| Corr-CL-V | 0.0148 | 21.6 | |

Residual variability

| Parameter | Estimate | RSE (%) |
|---|---|---|
| Proportional term | 0.182 | 5.70 |
| Additive term (IU/dL) | 0.837 | 39.4 |

Table 3 Abbreviations are: CL, clearance from central compartment; CV, coefficient of variation; popPK, population pharmacokinetic; RSE (%), percentage of relative standard error (SE) (100% × SE/estimate); V, volume of central compartment; ⊖ is the population estimate of PK parameter.

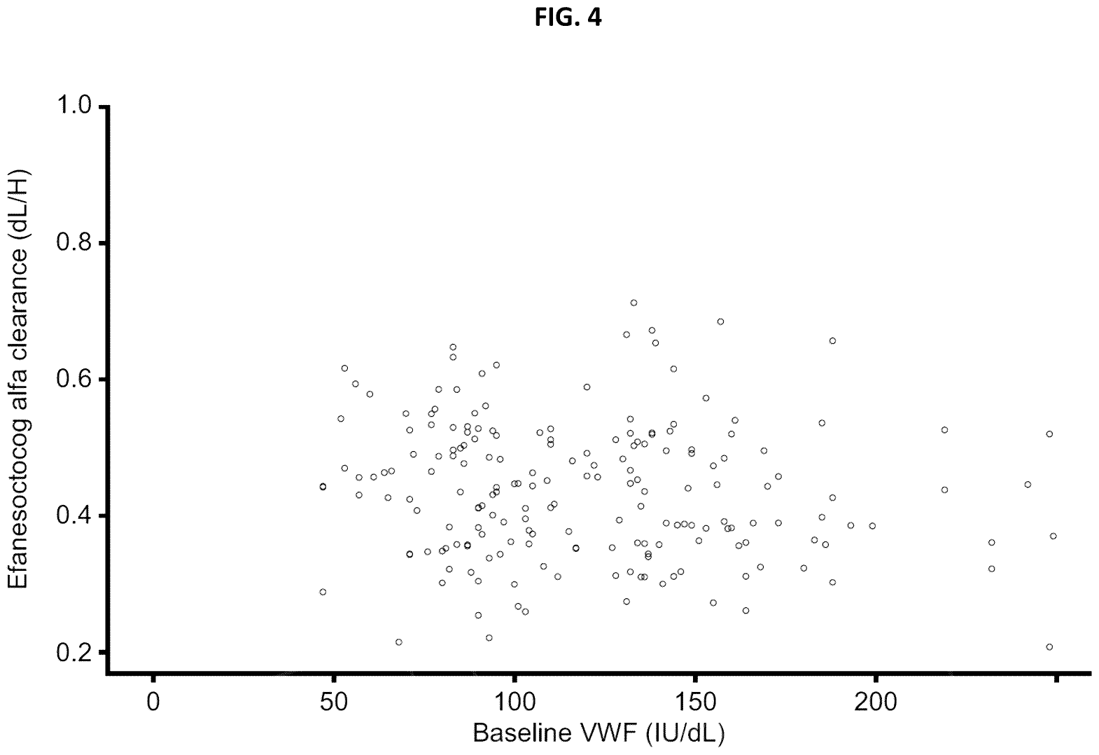

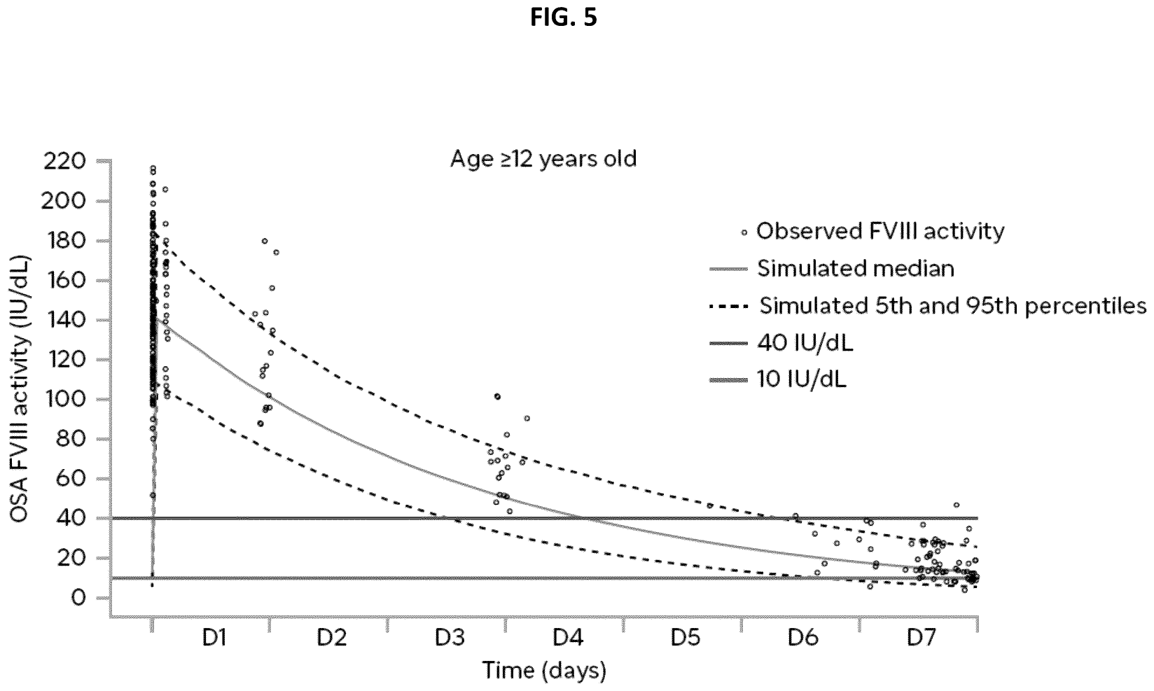

The final popPK model described the FVIII activity over time profile, captured inter-individual variability in FVIII activity, and precisely estimated moderate inter-individual variability in CL and V (Table 3). Body weight effect allometric exponents showed that CL and V increase with body weight, with overall faster elimination with lower body weight. Asian race was identified as a statistically significant covariate on CL (P<0.001); CL in Asians was 10.4% lower than in non-Asians. Baseline VWF level was not identified as a statistically significant covariate in the final popPK model, consistent with prior studies that demonstrate that the PK of efanesoctocog alfa is VWF-independent. For example, FIG. 4 shows the independence of efanesoctocog alfa clearance on baseline VWF levels in adult and adolescent patients. Blood type was not identified as a statistically significant covariate in the final PopPK model. Simulated steady-state FVIII activity over time for efanesoctocog alfa and population predicted and individual predicted versus observed FVIIII activity in the final PopPK model is presented in FIG. 5, which illustrated FVIII activity >40 IU/dL for 3 to 4 days post dose.

The final popPK model showed that a once-weekly efanesoctocog alfa (50 IU/kg) prophylaxis regimen achieves a steady state C_{trough} of >10 IU/dl and the time to 40 IU/dL FVIII activity was 3 to 4 days in the majority of adult and adolescent patients, irrespective of body weight and race. Simulations for perioperative management during major surgery and treatment of major bleeds showed that a loading dose of 50 IU/kg, followed by 30 IU/kg every 2-3 days in the postoperative period, met the World Federation of Hemophilia guidelines for peak FVIII activity for most adults and adolescents. Similarly, for minor surgeries and treatment of moderate to minor bleeds, a single dose of 50 IU/kg efanesoctocog alfa resulted in peak FVIII (>50 IU/dl to 80 IU/dL) activity that met these guidelines.

Conclusions: A linear one-compartment popPK model was able to adequately characterize FVIII activity in patients with severe hemophilia A. Although CL and V depended on body weight and Asian race was identified as a covariate on CL, body weight and Asian race's limited influence on FVIII exposure was not considered clinically meaningful. PopPK simulations demonstrated that 50 IU/kg once weekly efanesoctocog alfa achieved sustained FVIII activity in the normal to near-normal range (>40 IU/dL) for 3-4 days and >10 IU/dl at Day 7 in most adults and adolescents. PopPK simulations also supported the Phase 3 dose regimens selected for regular prophylaxis, treatment of bleeds, and perioperative management. Efanesoctocog alfa individual clearance was independent of baseline VWF in adults and adolescents.

Additional Details Relating to the Development of the popPK Model

Available data from adult, adolescent, and pediatric phase 3 studies with efanesoctocog alfa were incorporated in development of the population pharmacokinetic (PopPK) model. Complete data from adult and adolescent study and partial data from pediatric and long-term safety studies were also included.

TABLE 4
Description of studies used in the PopPK analysis.

| | | BIVV001 Dose | Duration | | |
|---|---|---|---|---|---|
| Phase | Study | Regimens | of Treatment | Population | N^{a} |
| 1/2a | TDU16220 | IV 25 and | Single dose | Adtil (≥18 year) | 15 |
| | | 65 IU/kg | | Hemophilia A | |
| | | | | patients | |
| 1/2a | TDR16219 | IV 50 and | Multiple dose. | Adult (≥18 year) | 24 |
| | | 65 IU/kg | QW for 4 weeks | Hemophilia A | |
| | | | | patients | |
| 3 | EFC16293 | IV 50 IU/kg | Multiple dose. | Adult and adolescent | 159^{b} |
| | | | QW for 52 weeks | (≥12 year) | |
| | | | | Hemophilia A | |
| | | | | patients | |
| 3 | EFC16295 | IV 50 IU/kg | Multiple dose. | Pediatric (<12 year) | 61 |
| | | | QW for 52 weeks | Hemophilia A | |
| | | | | patients | |
| 3 | LTS16294 | IV 50 IU/kg | Multiple dose. | Adult, adolescent, | 3^{c} |
| | | | QW for 52 weeks | and pediatric | |
| | | | | Hemophilia A | |
| | | | | patients | |

^{a}Number exposed to BIVV001 in each study for PopPK model development; total N = 260, with 199 actuts/adolescent patients and 61 pediatric patients from EFC15295. 1 patient from EFC16295 did not have dosing information and was excluded.
^{b}In EFC16293. 17 patients are in sequential arm, in which patients skip the dose on week 1 day 7 and week 26 day 7, to allow estimation of terminal hall-life by collection of FVIII activity samples upto day 15 alter day 1 and week 26 dose.
^{c}3 patients (2 from Arm A and 1 from Arm B) who had surgery in the LTS16294 study are included in the PopPK model development. 2 of the 3 patients continued from EFC16293.

Table 4 Definitions: a) Number exposed to efanesoctocog alfa in each study for PopPK model development; total N=260, with 199 adults & adolescent patients and 61 pediatric patients from EFC15295; b) Only patients who have surgery in (3 patients) the LTS16294 study are included in the PopPK model development; c) In EFC16293, 17 patients are in sequential arm, in which patients skip the dose on week 1 day 7 and week 26 day 7, to allow estimation of terminal half-life by collection of one stage clotting (OSC) FVIII activity samples up to day 15 after day 1 and week 26 dose.

Phase 1 PopPK analysis described the OSC FVIII activity data using 1-CMT model with bodyweight as a covariate on CL, V and level of hematocrit as covariate on V. In the final PopPK analysis, the 1-CMT model is chosen to be the structural model to describe OSC FVIII activity profile, and the base model includes WT effect. Further covariate screening was done on base POP PK model.

Bodyweight in kg (WT), or other continuous covariates, was scaled to median baseline WT (median baseline value) in adults and adolescents (78.3 kg) for evaluating as adult covariate effect or pediatric allometry effect.

For example, CL=TVCL*(WT_{time-varying}/78.3)_{CLexp}*(exp(ETA1)), for adult covariate effect and pediatric allometry effect. A similar approach was used for volume.

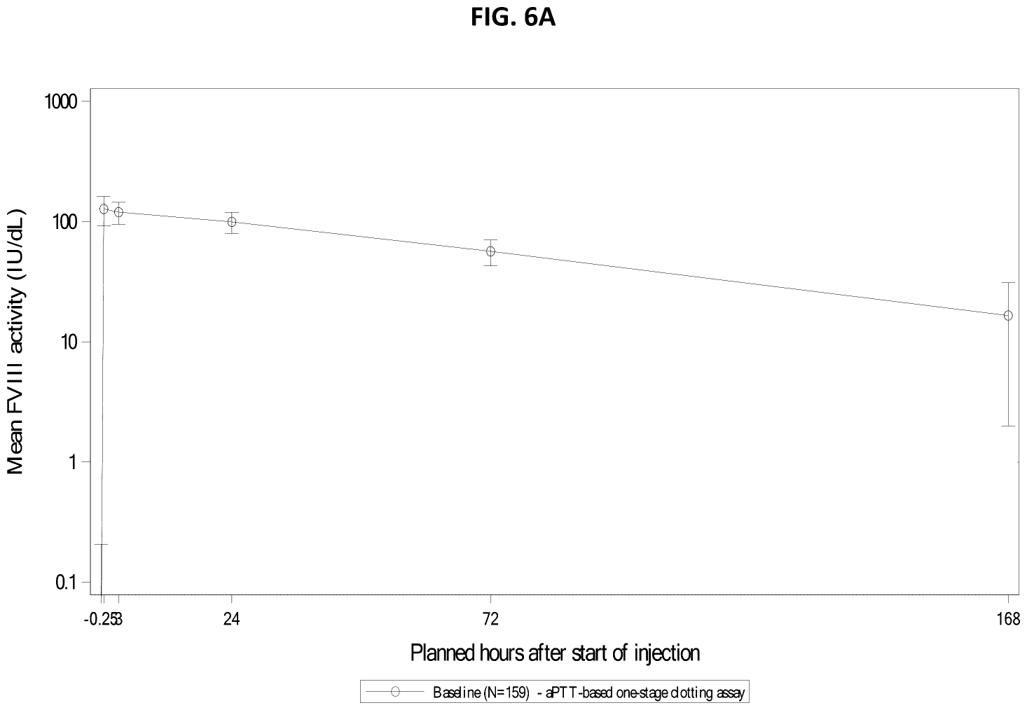

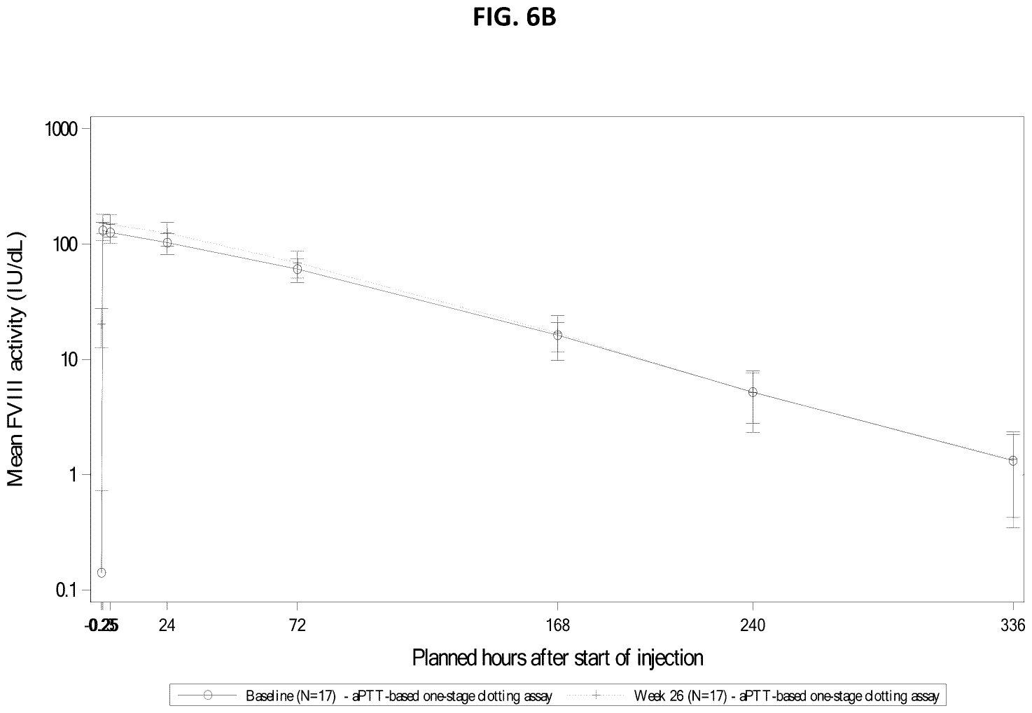

Datasets: One popPK dataset for observed data is based on the adult/adolescent study EFC16293. FIGS. 6A and 6B show a baseline corrected FVIII activity time profile. Day 1 (baseline) is shown for all patients (FIG. 6A). Day 1 (baseline) and at Week 26 is shown for sequential arm patients (FIG. 6B). The FVIII activity time profile follows a general one-compartment (linear decline on log-scale) type kinetics. The FVIII activity shows a mean half life for efanesoctocog alfa at 47.8 hours.

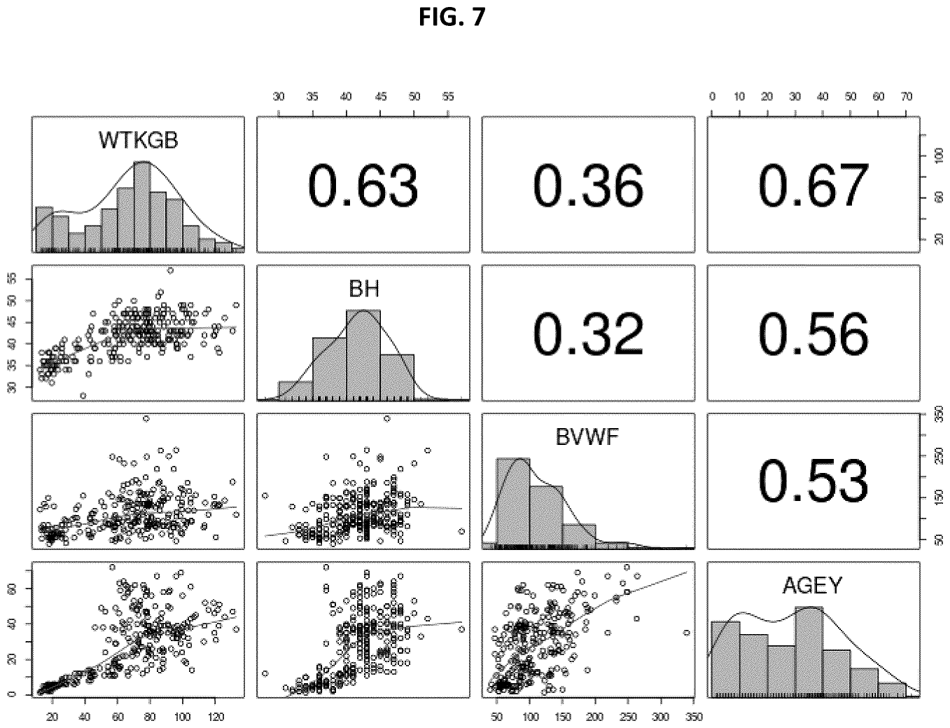

The correlation between 4 continuous covariates at baseline is shown in FIG. 7. Baseline weight (WTKGB) had a median of 78.3, which excluded the EFC16295 study. Baseline race (BH) had a median of 43 (also excluding the EFC16295 study). Baseline VWF (BVWF) showed a median of 112 (also excluding the EFC16295 study). Age appeared to be correlated to WT, so age is not tested as a covariate. Race and VWF were tested as covariates and found not to be significant.

The distribution of categorical covariates such as blood type, race, HIV status, and HCV status is shown in Table 5. Data in table 5 includes all studies (n=260). Black (1.92%) and Other race (3.08%) are present in about 5% of patients. For blood types, blood type A & O have more patients, 29.62% and 36.15% respectively. Blood type B is present in <10% (9.23%) of patients. Blood type AB is present in <5% (3.85%) of patients. HCV and HIV positive patients are older with no pediatric patients being HCV or HIV positive. There are 2 patients with Age <2 years. Out of the categorical factors shown, only HCV status, HIV status, blood type (A, B and O), and Race (Caucasian and Asian) were tested as covariates.

TABLE 5
Descriptive statistics of the catgorical covariates

| Study | N | Covariate | Type | n (%) |
|---|---|---|---|---|
| All | 260 | Gender | Female | 1 (0.4) |
| | | | Male | 259 (89.6) |
| All | 260 | Age group | Adult | 173 (66.5) |
| | | | (18 years ≤ Age) | |
| | | | Adolescent (12 years ≤ | 26 (10) |
| | | | Age < 18 years) | |
| | | | Older pediatric (6 years ≤ | 35 (13.5) |
| | | | Age < 12 years) | |
| | | | Younger pediatric | 26 (10) |
| | | | (Age < 6 years) | |
| All | 260 | Hepatitis C | With | 76 (29.2) |
| | | infection status | Without | 184 (70.8) |
| All | 260 | HIV infection | With | 24 (9.2) |
| | | status | Without | 236 (90.8) |
| All | 260 | Race | Caucasian | 177 (68.1) |
| | | | Asian | 40 (15.4) |
| | | | Black | 6 (1.92) |
| | | | Other race | 8 (3.08) |
| | | | Race not reported | 30 (11.5) |
| All | 260 | Blood type | Blood type A | 77 (29.62) |
| | | | Blood type AB | 10 (3.85) |
| | | | Blood type B | 24 (9.23) |
| | | | Blood type O | 94 (36.15) |
| | | | Blood type unknown | 55 (21.15) |

OSC activity is concentration (C) in central compartment. All parameters for base and final covariate model were estimated with acceptable precision. Adding bodyweight effect decreased the instrumental variables estimation (IIV) on CL and V, while adding the Asian race effect on CL decreased the IIV on CL. The exponents for WT effect on CL and V are acceptable when compared to the simple allometry exponents. Asian race effect was identified on CL, with clearance for Asians 10.4% lower than non-Asians of identical bodyweight.

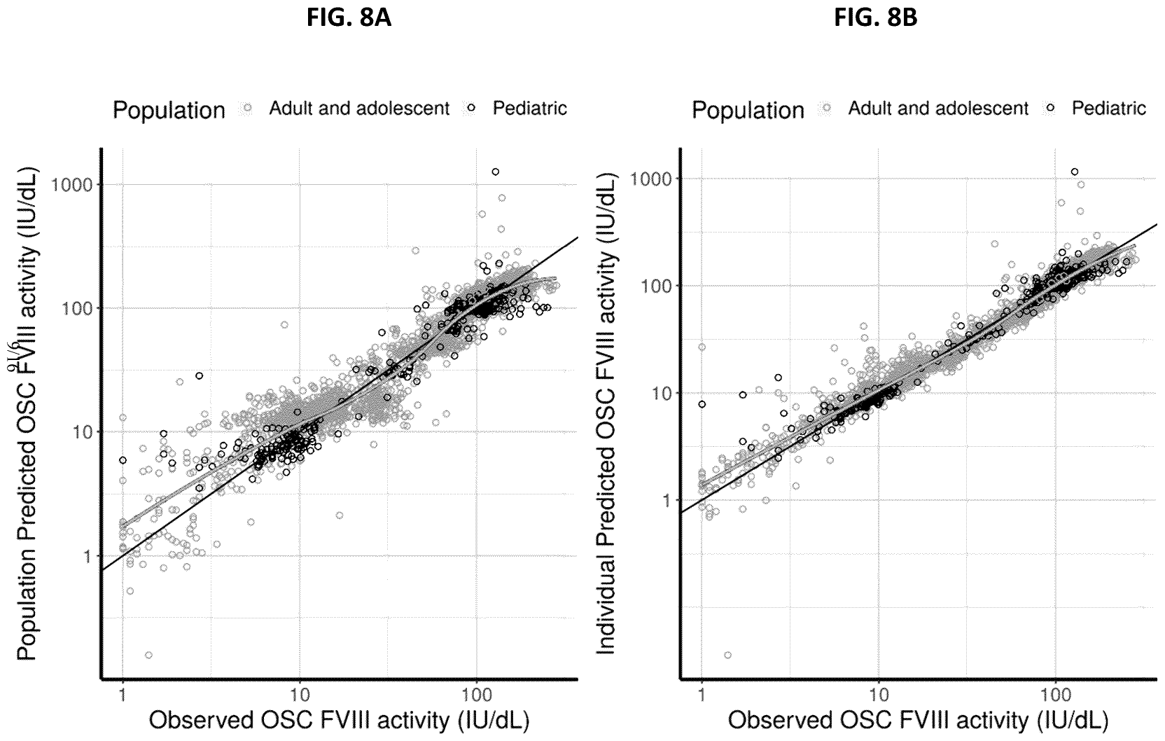

FIGS. 8A and 8B show population predictions (PRED) and individual predictions (IPRED) versus DV, respectively. This demonstrates that the population model and individual model are able to describe the PK data across the age categories.

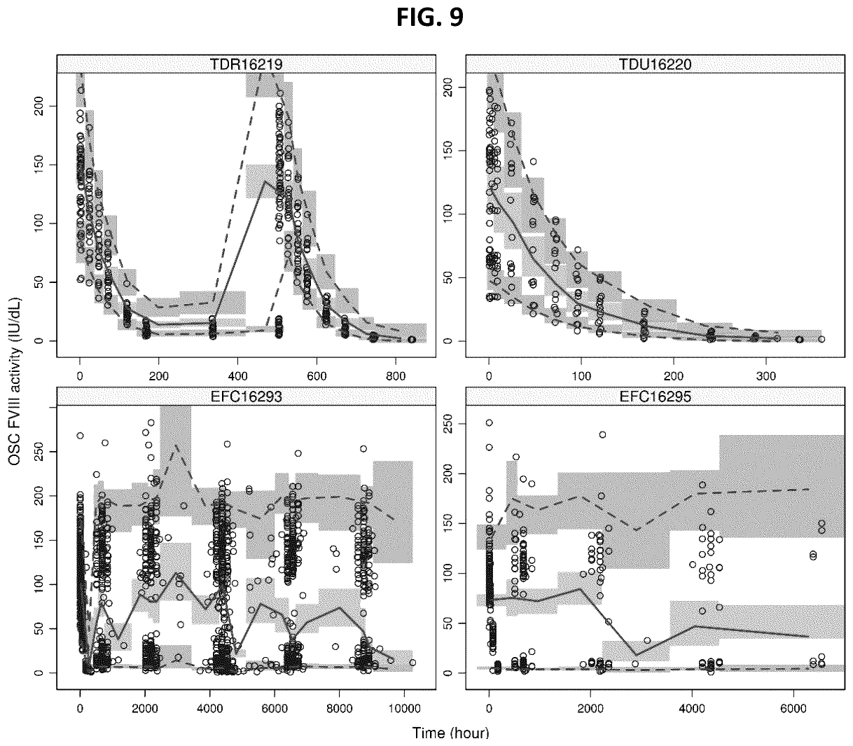

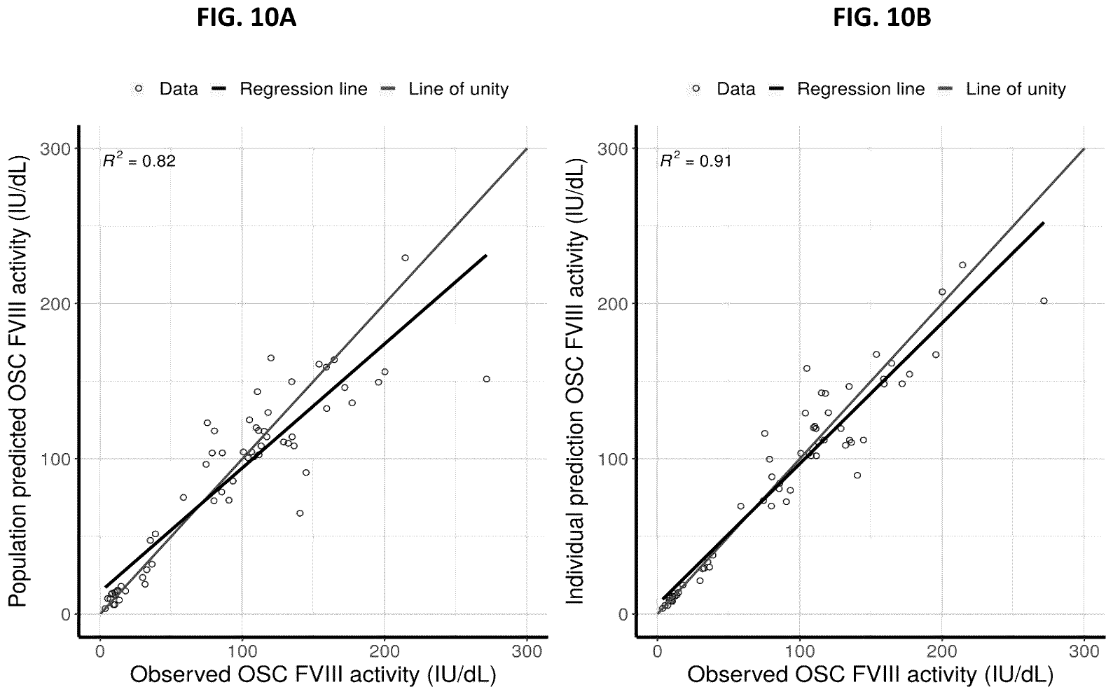

FIG. 9 demonstrates visual predictive checks (VPC) for the final PopPK model. The VPC for each study show that a large majority of the observed FVIII activity data were within in the prediction range [5th-95th percentiles]. For the purpose of VPC, the one unique patient from LTS16294 was considered in EFC16293. FIGS. 10A and 10B demonstrate population predictions (PRED) and individual predictions (IPRED) versus DV, respectively, for surgery. Data from 19 patients from EFC16293, EFC16295, and LTS16294 is included, during the surgery time frame. The model performs reasonably well in describing PK data collected during surgery and after ad hoc surgery dosing.

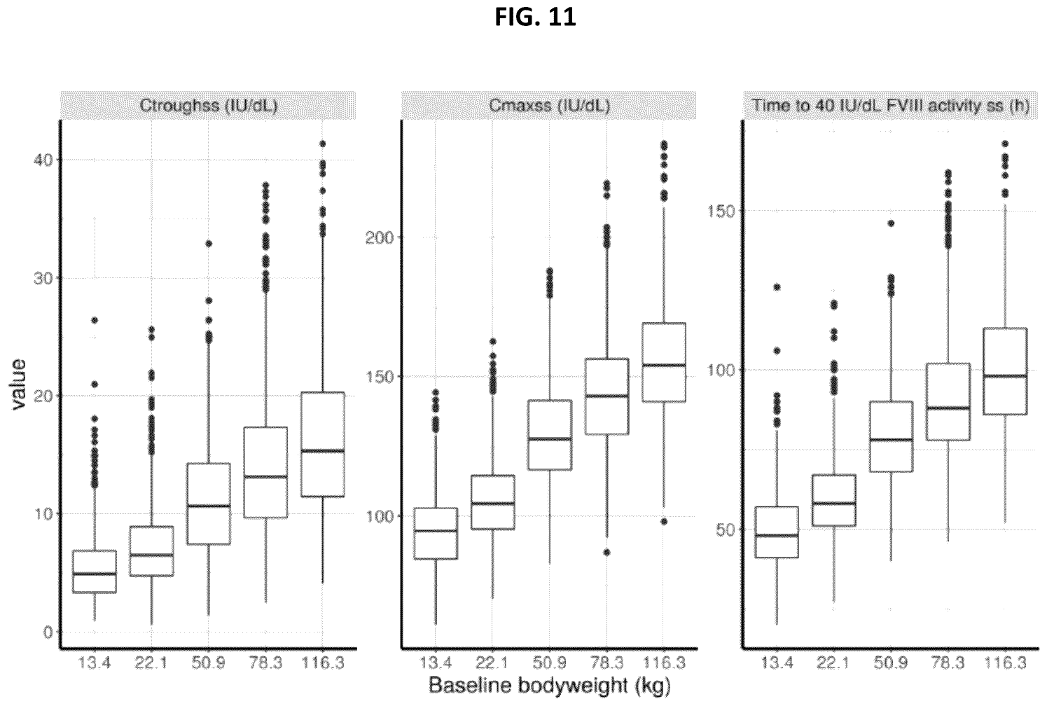

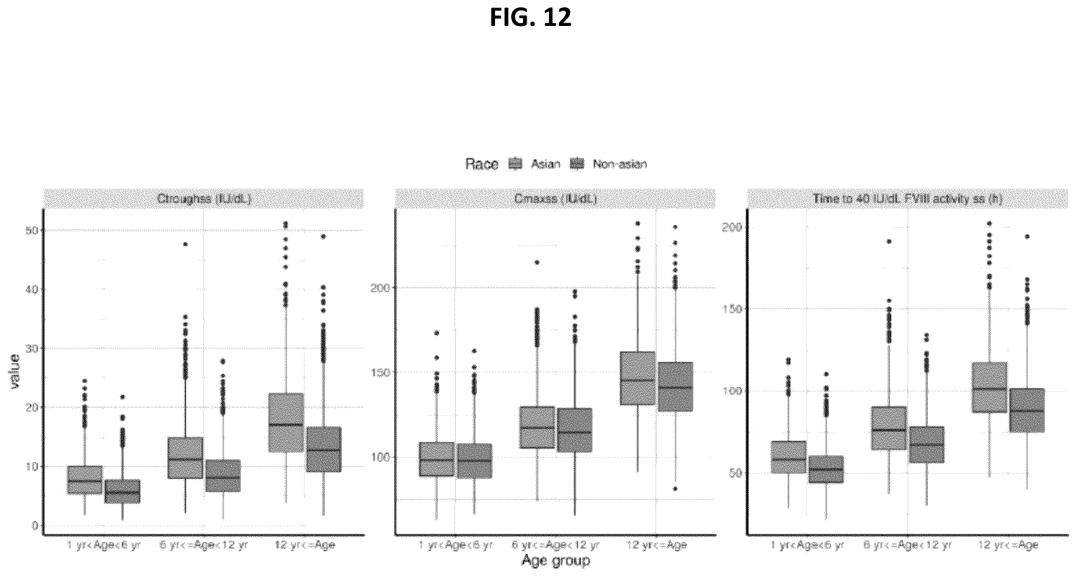

FIG. 11 shows the distribution of steady state C_{trough}, Cₘₐₓₛₛ and time to 40 IU/dL FVIII activity across all populations according to baseline body weight (kg). FIG. 12 shows the distribution of steady state C_{trough}, Cₘₐₓₛₛ and time to 40 IU/dL across non-Asian and Asian populations for all age groups. The steady state FVIII activity Cₘₐₓ, C_{trough} and time to 40 IU/dL FVIII activity increases with increasing body weight and is higher in Asians compared to Non-Asians. However, regardless of bodyweight and race, the 50 IU/kg QW prophylaxis regimen showed that a steady state C_{trough}>10 IU/dl and time to 40 IU/dL FVIII activity of 3 to 4 days is achieved for the majority of the adult and adolescent (Age ≥12 yr) population. The 50 IU/kg QW prophylaxis regimen also showed that a steady state C_{trough}>5 IU/dL & Time to 40 IU/dL FVIII activity of 2 to 3 days is achieved for the majority of the pediatric (Age <12 yr) population.

Major surgeries and major bleeds: The model was analyzed with regard to major surgeries and major bleeds. Major surgeries and major bleeds were categorized based on the criteria listed in Table 6.

TABLE 6

| Day | Major Surgery | Major Bleed |
|---|---|---|
| Pre-operative | Peak > 80 IU −100 IU/dL | Peak > 80 IU −100 IU/dL |
| (Day of bleed) | | |
| Day 1 to day 3 | Peak > 60 IU −80 IU/dL | Peak > 50 IU/dL (day 3) |
| Day 4 to day 7 | Peak > 40 IU −60 IU/dL | Peak > 50 IU/dL |
| Day 7 to day 14 | Peak > 30 IU −50 IU/dL | Peak > 50 IU/dL |

For major surgeries and bleeds, simulation was based on a dosing regimen of a single dose at 50 IU/kg with additional doses of 30 or 50 IU/kg every 2 to 3 days if needed. Thus, 50 IU/kg Q2D, 50 IU/kg Q3D, 30 IU/kg Q2D and 30 IU/kg Q3D are possible combinations of a dosing regimen after the pre-operative dose of 50 IU/kg (QXD is every X days). These same simulations as can be applied to both major surgeries and major bleeds, as both comprise the same dosing combinations.

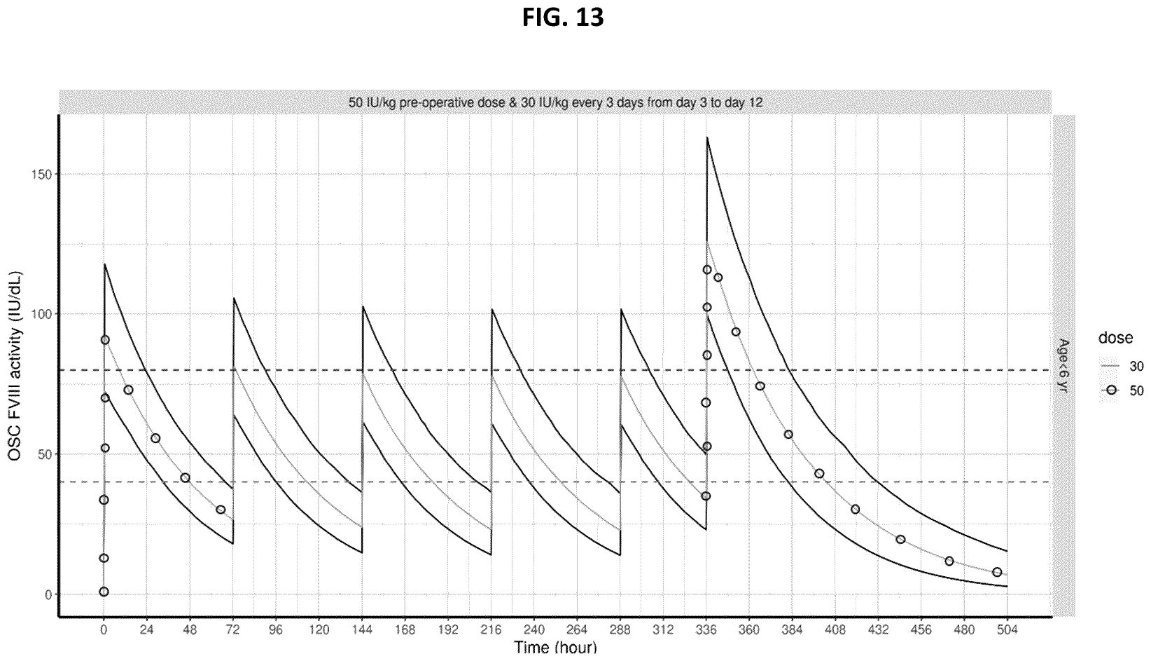

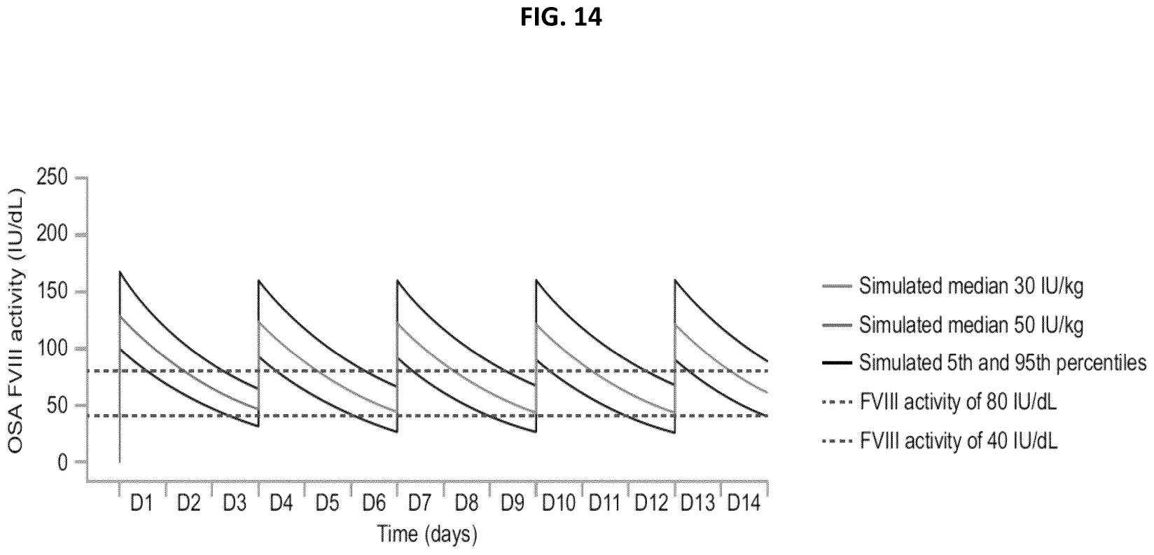

FIG. 13 shows the simulated OSC FVIII activity for major bleeds and major surgeries over time in subjects below six years of age. Across the entire surgery period, more than 95% of patients ages 6 and above meet the major surgery criteria, and more than 80% of patients under the age of 6 meet the surgery criteria, across the entire surgery period.

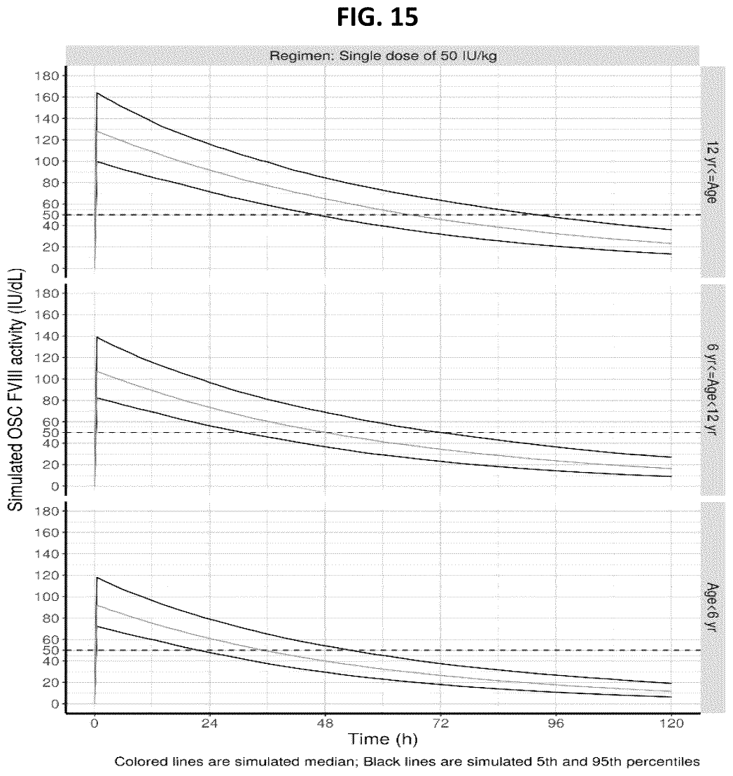

FIG. 14 shows the simulated FVIII activity for a dose of 50 IU/kg efanesoctocog alfa followed by 30 IU/kg every 3 days until Day 14 in a virtual adult and adolescent population. The simulations illustrated that an initial dose of 50 IU/kg, followed by 30 IU/kg every 3 days until Day 14, should be sufficient for perioperative management during major surgery (FIG. 15), as well as for treatment of major bleeds. >95% of adult and adolescent patients are predicted to meet the World Federation of Hemophilia (WFH) guidelines (Srivastava A, et al. Haemophilia. 2020; 26 Suppl 6:1-158) for peak FVIII activity (>80-100 IU/dL preoperative or on day of major bleed). In addition, other dosing regimens such as an initial dose of 50 IU/kg followed by 50 or 30 IU/kg every 2 or 3 days were simulated, and these additional dosing regimens were also predicted to meet the WFH peak FVIII guidelines. The simulations thus illustrated that an initial dose of 50 IU/kg, followed by 50 IU/kg or 30 IU/kg every 2 or 3 days, would meet the WFH peak FVIII guidelines in the majority (>95%) of adult and adolescent patients to manage during major surgery and treatment of major bleeds.

Minor surgeries and minor or moderate bleeds: FIG. 15 shows the simulated OSC FVIII activity over time for all age groups. More than 95% of patients in all age groups meet the criteria of peak FVIII >50 IU/dL after the pre-operative dose for minor surgery. Similarly, more than 95% patients in all age groups meet the criteria of peak FVIII >40 IU/dL as needed for minor/moderate bleed management. With additional doses of 30 or 50 IU/kg every 2 or 3 days, more than 95% patients in all age groups meet the criteria of peak FVIII >50 IU/dL.

Conclusions: Based on this data, the one compartment (1-CMT) model describes the adult, adolescent, and pediatric OSC FVIII activity data reasonably well. Body weight effect (on CL and V) was included in the base model while Asian race effect (on CL) was identified as a statistically significant covariate. Simulations for a range of body weights show that fixed regimen of 50 IU/kg QW provides high FVIII activity in adult, adolescents, and pediatric populations, regardless of body weight and race. Simulations using this model were also able to support and show potential efanesoctocog alfa dosing schemes for surgery and bleeding scenarios.

### EMBODIMENTS OF THE DISCLOSURE

The present disclosure includes (and is not limited to) the following exemplary embodiments:

Embodiment 1. A method for determining efanesoctocog alfa dosing information for an individual subject, the method comprising
receiving information specific to the subject and calculating the efanesoctocog alfa dosing information using a software-based system,
wherein the system is programmed to implement a one-compartment efanesoctocog alfa popPK model,
wherein the efanesoctocog alfa popPK model comprises body weight as a covariate, and wherein the efanesoctocog alfa popPK model does not comprise the level of VWF or level of hematocrit as covariates.

Embodiment 2. The method of embodiment 1, further comprising outputting, by the software-based system, the dosing information for the subject.

Embodiment 3. The method of embodiment 2, further comprising outputting, by the software-based system, a suggested dose regimen.

Embodiment 4. The method of any one of embodiments 1-3, wherein desired treatment outcome information is also received.

Embodiment 5. A method of estimating individualized efanesoctocog alfa dosing information for a subject, the method comprising:
(a) receiving, by a processing device, subject information and/or desired treatment outcome information by an application program programmed to operate with an efanesoctocog alfa popPK model, wherein the received information is transmitted by one or more electronic devices,
(b) calculating, using at least a software-based system, individualized efanesoctocog alfa dosing information using the efanesoctocog alfa popPK model and the received information, and
(c) transmitting the calculated individualized efanesoctocog alfa dosing information of (b) to one or more electronic devices for output of the information,
   wherein the efanesoctocog alfa popPK model comprises body weight as a covariate, and wherein the efanesoctocog alfa popPK model does not comprise the level of VWF or the level of hematocrit as covariates.

Embodiment 6. A method of estimating individualized efanesoctocog alfa dosing information for a subject, the method comprising:
(a) receiving subject information and/or desired treatment outcome information by one or more electronic devices,
(b) transmitting, by a processing device, the subject information and/or desired treatment outcome information to an application program, wherein the application is programmed to implement an efanesoctocog alfa popPK model,
(c) receiving from the application program, individualized efanesoctocog alfa dosing information calculated using the efanesoctocog alfa popPK model and the transmitted information of (b), and
(d) outputting, by the one or more electronic devices, the individualized efanesoctocog alfa dosing information,
   wherein the efanesoctocog alfa popPK model comprises body weight as a covariate, and wherein the efanesoctocog alfa popPK model does not comprise the level of VWF or level of hematocrit as covariates.

Embodiment 7. A method of providing an efanesoctocog alfa dosing regimen based on median popPK, the method comprising:
(a) receiving subject information and/or desired treatment outcome information by a software-based system comprising an efanesoctocog alfa popPK model,
(b) calculating, by the software-based system, median PK information using the efanesoctocog alfa popPK model and the received information, and
(c) outputting, by the software-based system, the median PK information,
   wherein the efanesoctocog alfa popPK model comprises body weight as a covariate, and wherein the efanesoctocog alfa popPK model does not comprise the level of VWF or level of hematocrit as covariates.

Embodiment 8. A method of providing an efanesoctocog alfa dosing regimen based on median efanesoctocog alfa popPK, the method comprising:
(a) receiving subject information and/or desired treatment outcome information by one or more electronic devices,
(b) transmitting, by a processing device, the subject information and/or desired treatment outcome information to an application program, wherein the application is programmed to implement an efanesoctocog alfa popPK model,
(c) receiving from the application program, median efanesoctocog alfa PK dosing information calculated using the efanesoctocog alfa popPK model and the received information, and
(d) outputting, by the one or more electronic devices, the median PK information,
   wherein the efanesoctocog alfa popPK model comprises body weight as a covariate, and wherein the efanesoctocog alfa popPK model does not comprise the level of VWF or level of hematocrit as covariates.

Embodiment 9. A method of providing an efanesoctocog alfa dosing regimen, the method comprising:
(a) receiving, by a processing device, subject information and/or desired treatment outcome information by an application program programmed to implement an efanesoctocog alfa population pharmacokinetic (popPK) model, wherein the received information is transmitted by one or more electronic devices,
(b) calculating, by the application program, individualized efanesoctocog alfa dosing information using the efanesoctocog alfa popPK model and the received information, and
(c) transmitting, by a processing device, the individualized efanesoctocog alfa dosing regimen calculated dosing information of (b) to one or more electronic devices for output of the information,
   wherein the efanesoctocog alfa popPK model comprises body weight as a covariate, and wherein the efanesoctocog alfa popPK model does not comprise the level of VWF or level of hematocrit as covariates.

Embodiment 10. A method of estimating individualized subject efanesoctocog alfa PK information, the method comprising:
(a) receiving subject information by a software-based system comprising an efanesoctocog alfa popPK model,
(b) estimating, by the software-based system, individualized subject efanesoctocog alfa PK information using the efanesoctocog alfa popPK model and the received information, and
(c) outputting, by the software-based system, the individualized subject efanesoctocog alfa PK information,
   wherein the efanesoctocog alfa popPK model comprises body weight as a covariate, and wherein the efanesoctocog alfa popPK model does not comprise the level of VWF or level of hematocrit as covariates.

Embodiment 11. A method of estimating individualized subject efanesoctocog alfa PK information, the method comprising:
(a) receiving, by one or more electronic devices, subject information,
(b) transmitting, by a processing device, the subject information to an application program, wherein the application is programmed to implement an efanesoctocog alfa popPK model, and wherein the application program generates individualized subject efanesoctocog alfa PK information using the efanesoctocog alfa popPK model and the transmitted information,
(c) receiving the individualized subject efanesoctocog alfa PK information from the application program, and
(d) outputting, by the one or more electronic devices, the individualized subject PK information,
   wherein the efanesoctocog alfa popPK model comprises body weight as a covariate, and wherein the efanesoctocog alfa popPK model does not comprise the level of VWF or level of hematocrit as covariates.

Embodiment 12. A method of estimating individualized subject efanesoctocog alfa PK information, the method comprising:
(a) receiving subject information by an application program that is programmed to implement an efanesoctocog alfa popPK model, wherein the received information is transmitted by one or more electronic devices,
(b) calculating, by the application program, individualized subject efanesoctocog alfa PK information of efanesoctocog alfa using the efanesoctocog alfa popPK model and the received information, and
(c) transmitting, by a processing device, the calculated individualized subject efanesoctocog alfa PK information of (b) to one or more one or more electronic devices, for output of the information,
   wherein the efanesoctocog alfa popPK model comprises body weight as a covariate, and wherein the efanesoctocog alfa popPK model does not comprise the level of VWF or level of hematocrit as covariates.

Embodiment 13. A method of estimating individualized subject efanesoctocog alfa PK information, the method comprising:
(a) receiving, by one or more electronic devices, information regarding individual body weight and (i) desired raise of plasma factor activity level following the dose or (ii) desired dose or desired dose interval,
(b) transmitting, by a processing device, the information of (a) to an application program, wherein the application is programmed to implement an efanesoctocog alfa popPK model,
(c) receiving from the web-based server and program, individualized subject efanesoctocog alfa PK information calculated using the efanesoctocog alfa popPK model and the transmitted information of (b), and
(d) outputting, by the one or more electronic devices, the calculated individualized subject efanesoctocog alfa PK information,
   wherein the efanesoctocog alfa popPK model comprises body weight as a covariate, and wherein the efanesoctocog alfa popPK model does not comprise the level of VWF or level of hematocrit as covariates.

Embodiment 14. The method of any one of embodiments 5-13, wherein the subject information includes the subject's body weight.

Embodiment 15. The method of any one of embodiments 5-14, wherein the subject information includes a baseline FVIII activity level for the subject.

Embodiment 16. The method of any one of embodiments 5-15, wherein the subject information includes the subject's self-reported race.

Embodiment 17. The method of any one of embodiments 5-16, wherein the subject information includes whether the subject self-identifies as being Asian.

Embodiment 18. The method of any one of embodiments 5-17, wherein the subject provides the subject information.

Embodiment 19. The method of any one of embodiments 5-17, wherein a healthcare professional provides the subject information.

Embodiment 20. The method of any one of embodiments 5-19, wherein the subject information does not include the subject's level of VWF or hematocrit.

Embodiment 21. The method of any one of embodiments 1-20, wherein the system is programmed to implement a one-compartment efanesoctocog alfa popPK model comprising body weight as covariates to calculate the dosing information, and wherein the efanesoctocog alfa popPK model does not comprise the level of VWF or hematocrit as covariates.

Embodiment 22. The method of any one of embodiments 1-6, 8-9, or 14-21, further comprising selecting a dosing regimen based on the dosing information.

Embodiment 23. The method of any one of embodiments 7-8 or 10-21, further comprising selecting a dosing regimen based on the PK information.

Embodiment 24. The method of embodiment 22 or 23, further comprising administering the efanesoctocog alfa to the subject according to the selected dosing regimen.

Embodiment 25. The method of any one of embodiments 1-6, 8-9, 14-22 or 24, wherein the dosing information comprises estimated or predicted FVIII activity levels over time after administration of the efanesoctocog alfa.

Embodiment 26. The method of any one of embodiments 7-9 or 14-25, wherein the dosing regimen is a prophylaxis regimen.

Embodiment 27. The method of any one of embodiments 7-9 or 14-25, wherein the dosing regimen is an on-demand regimen.

Embodiment 28. The method of any one of embodiments 7-9 or 14-25, wherein the dosing regimen is for perioperative management of bleeding.

Embodiment 29. The method of any one of embodiments 4-5, wherein the desired treatment outcome information comprises a desired FVIII activity level.

Embodiment 30. The method of embodiment 29, wherein the desired FVIII activity level comprises the minimum FVIII activity level between doses.

Embodiment 31. The method of embodiment 29, wherein the desired FVIII activity level comprises the minimum FVIII activity level at a time point.

Embodiment 32. The method of embodiment 31, wherein the time point is 1, 2, 3, 4, 5, 6, 7, 8, 9, 10, 11, 12, 13, or 14 days after an administration of efanesoctocog alfa.

Embodiment 33. The method of any one of embodiments 1-32, wherein the efanesoctocog alfa popPK model comprises self-reported race as a covariate.

Embodiment 34. The method of embodiment 1-32, wherein the efanesoctocog alfa popPK model comprises whether the subject self-reports as Asian as a covariate.

Embodiment 35. The method of embodiment 33 or 34, wherein the subject does not self-report as being Asian.

Embodiment 36. The method of embodiment 33 or 34, wherein the subject self-reports as being Asian.

Embodiment 37. The method of any one of embodiments 1-38, wherein the efanesoctocog alfa popPK model is the efanesoctocog alfa popPK model [A].

Embodiment 38. A method of treating hemophilia A in a subject in need thereof, comprising administering to the subject a dose regimen selected according to any one of embodiments 1-37.

Embodiment 39. A device or system comprising a processor configured to provide dosing or PK information according to the method any one of embodiments 1-38.

Embodiment 40. A data processing apparatus, device, or system comprising a processor configured to implement a one-compartment efanesoctocog alfa popPK model, wherein the efanesoctocog alfa popPK model comprises body weight as a covariate, and wherein the efanesoctocog alfa popPK model does not comprise the level of VWF or level of hematocrit as covariates.

Embodiment 41. The data processing apparatus, device, or system of embodiment 40, wherein the efanesoctocog alfa popPK model comprises self-reported race as a covariate.

Embodiment 42. The data processing apparatus, device, or system of embodiment 40, wherein the efanesoctocog alfa popPK model comprises whether the subject self-reports as Asian as a covariate.

Embodiment 43. The data processing apparatus, device, or system of any one of embodiments 40-42, which comprises a smart phone, a tablet computer, a personal digital assistant, a handheld computer, a laptop computer, or a smart watch.

Embodiment 44. The data processing apparatus, device, or system of any one of embodiments 40-42, which comprises a smart phone.

Embodiment 45. The data processing apparatus, device, or system of any one of embodiments 40-42, which comprises a smart watch.

Embodiment 46. The data processing apparatus, device, or system of any one of embodiments 40-45, wherein the processor is configured to implement efanesoctocog alfa popPK model [A].

Embodiment 47. A computer program comprising instructions which, when the program is executed by a computer, cause the computer to carry out the method of any one of embodiments 1-38.

Embodiment 48. A computer program comprising instructions which, when the program is executed by a computer, cause the computer to implement a one-compartment efanesoctocog alfa popPK model, wherein the efanesoctocog alfa popPK model comprises body weight as a covariate, and wherein the efanesoctocog alfa popPK model does not comprise the level of VWF or level of hematocrit as covariates.

Embodiment 49. The computer program of embodiment 48, wherein the efanesoctocog alfa popPK model comprises self-reported race as a covariate.

Embodiment 50. The computer program of embodiment 48, wherein the efanesoctocog alfa popPK model comprises whether the subject self-reports as Asian as a covariate.

Embodiment 51. The computer program of embodiment 48, wherein the efanesoctocog alfa popPK model is efanesoctocog alfa popPK model [A].

Embodiment 52. A computer-readable medium comprising instructions which, when executed by a computer, cause the computer to provide carry out the method of any one of embodiments 1-38.

Embodiment 53. A computer-readable medium comprising instructions which, when executed by a computer, cause the computer to implement a one-compartment efanesoctocog alfa popPK model, wherein the efanesoctocog alfa popPK model comprises body weight as a covariate, and wherein the efanesoctocog alfa popPK model does not comprise the level of VWF or level of hematocrit as covariates.

Embodiment 54. The computer-readable medium of embodiment 53, wherein the efanesoctocog alfa popPK model comprises self-reported race as a covariate.

Embodiment 55. The computer-readable medium of embodiment 53, wherein the efanesoctocog alfa popPK model comprises whether the subject self-reports as Asian as a covariate.

Embodiment 56. The computer-readable medium of embodiment 53, wherein the efanesoctocog alfa popPK model is efanesoctocog alfa popPK model [A].

Embodiment 57. The method of embodiment 37, wherein the estimate of typical clearance (TVCL) in the efanesoctocog alfa popPK model [A] is 0.433 dL/h.

Embodiment 58. The method of embodiment 37 or 57, wherein the estimate of typical volume (TVV) in the efanesoctocog alfa popPK model [A] is 30.2 dL.

Embodiment 59. The method of any one of embodiments 37, 57, or 58, wherein the variability on clearance from central compartment (η₁) in the efanesoctocog alfa popPK model [A] is 0.0354.

Embodiment 60. The method of any one of embodiments 37 or 57-59, wherein the variability on volume of central compartment (η₂) in the efanesoctocog alfa popPK model [A] is 0.0209.

Embodiment 61. The data processing apparatus, device, or system of embodiment 46, wherein the estimate of typical clearance (TVCL) in the efanesoctocog alfa popPK model [A] is 0.433 dL/h.

Embodiment 62. The data processing apparatus, device, or system of any one of embodiments 46 or 61, wherein the estimate of typical volume (TVV) in the efanesoctocog alfa popPK model [A] is 30.2 dL.

Embodiment 63. The data processing apparatus, device, or system of any one of embodiments 46, 61, or 62, wherein the variability on clearance from central compartment (η1) in the efanesoctocog alfa popPK model [A] is 0.0354.

Embodiment 64. The data processing apparatus, device, or system of any one of embodiments 46 or 61-63, wherein the variability on volume of central compartment (η2) in the efanesoctocog alfa popPK model [A] is 0.0209.

Embodiment 65. The computer program of embodiment 51, wherein the estimate of typical clearance (TVCL) in the efanesoctocog alfa popPK model [A] is 0.433 dL/h.

Embodiment 66. The computer program of embodiment 51 or 65, wherein the estimate of typical volume (TVV) in the efanesoctocog alfa popPK model [A] is 30.2 dL.

Embodiment 67. The computer program of any one of embodiments 51, 65, or 66, wherein the variability on clearance from central compartment (η1) in the efanesoctocog alfa popPK model [A] is 0.0354.

Embodiment 68. The computer program of any one of embodiments 51 or 65-67, wherein the variability on volume of central compartment (η2) in the efanesoctocog alfa popPK model [A] is 0.0209.

Embodiment 69. The computer-readable medium of any one of embodiments 56, wherein the estimate of typical clearance (TVCL) in the efanesoctocog alfa popPK model [A] is 0.433 dL/h.

Embodiment 70. The computer-readable medium of any one of embodiments 56 or 69, wherein the estimate of typical volume (TVV) in the efanesoctocog alfa popPK model [A] is 30.2 dL.

Embodiment 71. The computer-readable medium of any one of embodiments 56, 69, or 70, wherein the variability on clearance from central compartment (η1) in the efanesoctocog alfa popPK model [A] is 0.0354.

Embodiment 72. The computer-readable medium of any one of embodiments 56 or 69-71, wherein the variability on volume of central compartment (η2) in the efanesoctocog alfa popPK model [A] is 0.0209.

TABLE 7
Efanesoctocog alfa Sequence Information

| Sequence | | |
|---|---|---|
| Description/ | | |
| SEQ ID NO. | Amino acid or nucleic acid sequence | |
| rFVIIIFc-ELNN | MQIELSTCFF LCLLRFCFSA TRRYYLGAVE LSWDYMQSDL GELPVDARFP PRVPKSFPFN | 60 |
| amino acid | TSVVYKKTLF VEFTDHLFNI AKPRPPWMGL LGPTIQAEVY DTVVITLKNM ASHPVSLHAV | 120 |
| sequence, | GVSYWKASEG AEYDDQTSQR EKEDDKVFPG GSHTYVWQVL KENGPMASDP LCLTYSYLSH | 180 |
| including | VDLVKDLNSG LIGALLVCRE GSLAKEKTQT LHKFILLFAV FDEGKSWHSE TKNSLMQDRD | 240 |
| signal | AASARAWPKM HTVNGYVNRS LPGLIGCHRK SVYWHVIGMG TTPEVHSIFL EGHTFLVRNH | 300 |
| peptide | RQASLEISPI TFLTAQTLLM DLGQFLLFCH ISSHQHDGME AYVKVDSCPE EPQLRMKNNE | 360 |
| (SEQ ID NO: 1) | EAEDYDDDLT DSEMDVVRFD DDNSPSFIQI RSVAKKHPKT WVHYIAAEEE DWDYAPLVLA | 420 |
| | PDDRSYKSQY LNNGPQRIGR KYKKVRFMAY TDETFKTREA IQHESGILGP LLYGEVGDTL | 480 |
| | LIIFKNQASR PYNIYPHGIT DVRPLYSRRL PKGVKHLKDF PILPGEIFKY KWTVTVEDGP | 540 |
| | TKSDPRCLTR YYSSFVNMER DLASGLIGPL LICYKESVDQ RGNQIMSDKR NVILFSVFDE | 600 |
| | NRSWYLTENI QRFLPNPAGV QLEDPEFQAS NIMHSINGYV FDSLQLSVCL HEVAYWYILS | 660 |
| | IGAQTDFLSV FFSGYTFKHK MVYEDTLTLF PFSGETVFMS MENPGLWILG CHNSDFRNRG | 720 |
| | MTALLKVSSC DKNTGDYYED SYEDISAYLL SKNNAIEPRS FSQNGTSESA TPESGPGSEP | 780 |
| | ATSGSETPGT SESATPESGP GSEPATSGSE TPGTSESATP ESGPGTSTEP SEGSAPGSPA | 840 |
| | GSPTSTEEGT SESATPESGP GSEPATSGSE TPGTSESATP ESGPGSPAGS PTSTEEGSPA | 900 |
| | GSPTSTEEGT STEPSEGSAP GTSESATPES GPGTSESATP ESGPGTSESA TPESGPGSEP | 960 |
| | ATSGSETPGS EPATSGSETP GSPAGSPTST EEGTSTEPSE GSAPGTSTEP SEGSAPGSEP | 1020 |
| | ATSGSETPGT SESATPESGP GTSTEPSEGS APASSEITRT TLQSDQEEID YDDTISVEMK | 1080 |
| | KEDFDIYDED ENQSPRSFQK KTRHYFIAAV ERLWDYGMSS SPHVLRNRAQ SGSVPQFKKV | 1140 |
| | VFQEFTDGSF TQPLYRGELN EHLGLLGPYI RAEVEDNIMV TFRNQASRPY SFYSSLISYE | 1200 |
| | EDQRQGAEPR KNFVKPNETK TYFWKVQHHM APTKDEFDCK AWAYFSDVDL EKDVHSGLIG | 1260 |
| | PLLVCHTNTL NPAHGRQVTV QEFALFFTIF DETKSWYFTE NMERNCRAPC NIQMEDPTFK | 1320 |
| | ENYRFHAING YIMDTLPGLV MAQDQRIRWY LLSMGSNENI HSIHFSGHVF TVRKKEEYKM | 1380 |
| | ALYNLYPGVF ETVEMLPSKA GIWRVECLIG EHLHAGMSTL FLVYSNKCQT PLGMASGHIR | 1440 |
| | DFQITASGQY GQWAPKLARL HYSGSINAWS TKEPFSWIKV DLLAPMIIHG IKTQGARQKF | 1500 |
| | SSLYISQFII MYSLDGKKWQ TYRGNSTGTL MVFFGNVDSS GIKHNIFNPP IIARYIRLHP | 1560 |
| | THYSIRSTLR MELMGCDLNS CSMPLGMESK AISDAQITAS SYFTNMFATW SPSKARLHLQ | 1620 |
| | GRSNAWRPQV NNPKEWLQVD FQKTMKVTGV TTQGVKSLLT SMYVKEFLIS SSQDGHQWTL | 1680 |
| | FFQNGKVKVF QGNQDSFTPV VNSLDPPLLT RYLRIHPQSW VHQIALRMEV LGCEAQDLYD | 1740 |
| | KTHTCPPCPA PELLGGPSVF LFPPKPKDTL MISRTPEVTC VVVDVSHEDP EVKFNWYVDG | 1800 |
| | VEVHNAKTKP REEQYNSTYR VVSVLTVLHQ DWLNGKEYKC KVSNKALPAP IEKTISKAKG | 1860 |
| | QPREPQVYTL PPSRDELTKN QVSLTCLVKG FYPSDIAVEW ESNGQPENNY KTTPPVLDSD | 1920 |
| | GSFFLYSKLT VDKSRWQQGN VFSCSVMHEA LHNHYTQKSL SLSPG | 1965 |
| rFVIIIFc-ELNN | atgcaaatag agctctccac ctgcttcttt ctgtgccttt tgcgattctg ctttagtgcc | 60 |
| nucleotide | accagaagat actacctggg tgcagtggaa ctgtcatggg actatatgca aagtgatctc | 120 |
| sequence | ggtgagctgc ctgtggacgc aagatttcct cctagagtgc caaaatcttt tccattcaac | 180 |
| (SEQ ID NO: 2) | acctcagtcg tgtacaaaaa gactctgttt gtagaattca cggatcacct tttcaacatc | 240 |
| | gctaagccaa ggccaccctg gatgggtctg ctaggtccta ccatccaggc tgaggtttat | 300 |
| | gatacagtgg tcattacact taagaacatg gcttcccatc ctgtcagtct tcatgctgtt | 360 |
| | ggtgtatcct actggaaagc ttctgaggga gctgaatatg atgatcagac cagtcaaagg | 420 |
| | gagaaagaag atgataaagt cttccctggt ggaagccata catatgtctg gcaggtcctg | 480 |
| | aaagagaatg gtccaatggc ctctgaccca ctgtgcctta cctactcata tctttctcat | 540 |
| | gtggacctgg taaaagactt gaattcaggc ctcattggag ccctactagt atgtagagaa | 600 |
| | gggagtctgg ccaaggaaaa gacacagacc ttgcacaaat ttatactact ttttgctgta | 660 |
| | tttgatgaag ggaaaagttg gcactcagaa acaaagaact ccttgatgca ggatagggat | 720 |
| | gctgcatctg ctcgggcctg gcctaaaatg cacacagtca atggttatgt aaacaggtct | 780 |
| | ctgccaggtc tgattggatg ccacaggaaa tcagtctatt ggcatgtgat tggaatgggc | 840 |
| | accactcctg aagtgcactc aatattcctc gaaggtcaca catttcttgt gaggaaccat | 900 |
| | cgccaggcgt ccttggaaat ctcgccaata actttcctta ctgctcaaac actcttgatg | 960 |
| | gaccttggac agtttctact gttttgtcat atctcttccc accaacatga tggcatggaa | 1020 |
| | gcttatgtca aagtagacag ctgtccagag gaaccccaac tacgaatgaa aaataatgaa | 1080 |
| | gaagcggaag actatgatga tgatcttact gattctgaaa tggatgtggt caggtttgat | 1140 |
| | gatgacaact ctccttcctt tatccaaatt cgctcagttg ccaagaagca tcctaaaact | 1200 |
| | tgggtacatt acattgctgc tgaagaggag gactgggact atgctccctt agtcctcgcc | 1260 |
| | cccgatgaca gaagttataa aagtcaatat ttgaacaatg gccctcagcg gattggtagg | 1320 |
| | aagtacaaaa aagtccgatt tatggcatac acagatgaaa cctttaagac tcgtgaagct | 1380 |
| | attcagcatg aatcaggaat cttgggacct ttactttatg gggaagttgg agacacactg | 1440 |
| | ttgattatat ttaagaatca agcaagcaga ccatataaca tctaccctca cggaatcact | 1500 |
| | gatgtccgtc ctttgtattc aaggagatta ccaaaaggtg taaaacattt gaaggatttt | 1560 |
| | ccaattctgc caggagaaat attcaaatat aaatggacag tgactgtaga agatgggcca | 1620 |
| | actaaatcag atcctcggtg cctgacccgc tattactcta gtttcgttaa tatggagaga | 1680 |
| | gatctagctt caggactcat tggccctctc ctcatctgct acaaagaatc tgtagatcaa | 1740 |
| | agaggaaacc agataatgtc agacaagagg aatgtcatcc tgttttctgt atttgatgag | 1800 |
| | aaccgaagct ggtacctcac agagaatata caacgctttc tccccaatcc agctggagtg | 1860 |
| | cagcttgagg atccagagtt ccaagcctcc aacatcatgc acagcatcaa tggctatgtt | 1920 |
| | tttgatagtt tgcagttgtc agtttgtttg catgaggtgg catactggta cattctaagc | 1980 |
| | attggagcac agactgactt cctttctgtc ttcttctctg gatatacctt caaacacaaa | 2040 |
| | atggtctatg aagacacact caccctattc ccattctcag gagaaactgt cttcatgtcg | 2100 |
| | atggaaaacc caggtctatg gattctgggg tgccacaact cagactttcg gaacagaggc | 2160 |
| | atgaccgcct tactgaaggt ttctagttgt gacaagaaca ctggtgatta ttacgaggac | 2220 |
| | agttatgaag atatttcagc atacttgctg agtaaaaaca atgccattga accaagaagc | 2280 |
| | ttctctcaaa acggtacctc agagtctgct acccccgagt cagggccagg atcagagcca | 2340 |
| | gccacctccg ggtctgagac acccgggact tccgagagtg ccacccctga gtccggaccc | 2400 |
| | gggtccgagc ccgccacttc cggctccgaa actcccggca caagcgagag cgctacccca | 2460 |
| | gagtcaggac caggaacatc tacagagccc tctgaaggct ccgctccagg gtccccagcc | 2520 |
| | ggcagtccca ctagcaccga ggagggaacc tctgaaagcg ccacacccga atcagggcca | 2580 |
| | gggtctgagc ctgctaccag cggcagcgag acaccaggca cctctgagtc cgccacacca | 2640 |
| | gagtccggac ccggatctcc cgctgggagc cccacctcca ctgaggaggg atctcctgct | 2700 |
| | ggctctccaa catctactga ggaaggaacc tcaaccgagc catccgaggg atcagctccc | 2760 |
| | ggcacctcag agtcggcaac cccggagtct ggacccggaa cttccgaaag tgccacacca | 2820 |
| | gagtccggtc ccgggacttc agaatcagca acacccgagt ccggccctgg gtctgaaccc | 2880 |
| | gccacaagtg gtagtgagac accaggatca gaacctgcta cctcagggtc agagacaccc | 2940 |
| | ggatctccgg caggctcacc aacctccact gaggagggca ccagcacaga accaagcgag | 3000 |
| | ggctccgcac ccggaacaag cactgaaccc agtgagggtt cagcacccgg ctctgagccg | 3060 |
| | gccacaagtg gcagtgagac acccggcact tcagagagtg ccacccccga gagtggccca | 3120 |
| | ggcactagta ccgagccctc tgaaggcagt gcgccagcct cgagcgaaat aactcgtact | 3180 |
| | actcttcagt cagatcaaga ggaaattgac tatgatgata ccatatcagt tgaaatgaag | 3240 |
| | aaggaagatt ttgacattta tgatgaggat gaaaatcaga gcccccgcag ctttcaaaag | 3300 |
| | aaaacacgac actattttat tgctgcagtg gagaggctct gggattatgg gatgagtagc | 3360 |
| | tccccacatg ttctaagaaa cagggctcag agtggcagtg tccctcagtt caagaaagtt | 3420 |
| | gttttccagg aatttactga tggctccttt actcagccct tataccgtgg agaactaaat | 3480 |
| | gaacatttgg gactcctggg gccatatata agagcagaag ttgaagataa tatcatggta | 3540 |
| | actttcagaa atcaggcctc tcgtccctat tccttctatt ctagccttat ttcttatgag | 3600 |
| | gaagatcaga ggcaaggagc agaacctaga aaaaactttg tcaagcctaa tgaaaccaaa | 3660 |
| | acttactttt ggaaagtgca acatcatatg gcacccacta aagatgagtt tgactgcaaa | 3720 |
| | gcctgggctt atttctctga tgttgacctg gaaaaagatg tgcactcagg cctgattgga | 3780 |
| | ccccttctgg tctgccacac taacacactg aaccctgctc atgggagaca agtgacagta | 3840 |
| | caggaatttg ctctgttttt caccatcttt gatgagacca aaagctggta cttcactgaa | 3900 |
| | aatatggaaa gaaactgcag ggctccctgc aatatccaga tcgaagatcc cacttttaaa | 3960 |
| | gagaattatc gcttccatgc aatcaatggc tacataatgg atacactacc tggcttagta | 4020 |
| | atggctcagg atcaaaggat tcgatggtat ctgctcagca tgggcagcaa tgaaaacatc | 4080 |
| | cattctattc atttcagtgg acatgtgttc actgtacgaa aaaaagagga gtataaaatg | 4140 |
| | gcactgtaca atctctatcc aggtgttttt gagacagtgg aaatgttacc atccaaagct | 4200 |
| | ggaatttggc gggtggaatg ccttattggc gagcatctac atgctgggat gagcacactt | 4260 |
| | tttctggtgt acagcaataa gtgtcagact cccctgggaa tggcttctgg acacattaga | 4320 |
| | gattttcaga ttacagcttc aggacaatat ggacagtggg ccccaaagct ggccagactt | 4380 |
| | cattattccg gatcaatcaa tgcctggagc accaaggagc ccttttcttg gatcaaggtg | 4440 |
| | gatctgttgg caccaatgat tattcacggc atcaagaccc agggtgcccg tcagaagttc | 4500 |
| | tccagcctct acatctctca gtttatcatc atgtatagtc ttgatgggaa gaagtggcag | 4560 |
| | acttatcgag gaaattccac tggaacctta atggtcttct ttggcaatgt ggattcatct | 4620 |
| | gggataaaac acaatatttt taaccctcca attattgctc gatacatccg tttgcaccca | 4680 |
| | actcattata gcattcgcag cactcttcgc atggagttga tgggctgtga tttaaatagt | 4740 |
| | tgcagcatgc cattgggaat ggagagtaaa gcaatatcag atgcacagat tactgcttca | 4800 |
| | tcctacttta ccaatatgtt tgccacctgg tctccttcaa aagctcgact tcacctccaa | 4860 |
| | gggaggagta atgcctggag acctcaggtg aataatccaa aagagtggct gcaagtggac | 4920 |
| | ttccagaaga caatgaaagt cacaggagta actactcagg gagtaaaatc tctgcttacc | 4980 |
| | agcatgtatg tgaaggagtt cctcatctcc agcagtcaag atggccatca gtggactctc | 5040 |
| | ttttttcaga atggcaaagt aaaggttttt cagggaaatc aagactcctt cacacctgtg | 5100 |
| | gtgaactctc tagacccacc gttactgact cgctaccttc gaattcaccc ccagagttgg | 5160 |
| | gtgcaccaga ttgccctgag gatggaggtt ctgggctgcg aggcacagga cctctacgac | 5220 |
| | aaaactcaca catgcccacc gtgcccagct ccagaactcc tgggcggacc gtcagtcttc | 5280 |
| | ctcttccccc caaaacccaa ggacaccctc atgatctccc ggacccctga ggtcacatgc | 5340 |
| | gtggtggtgg acgtgagcca cgaagaccct gaggtcaagt tcaactggta tgtggacggc | 5400 |
| | gtggaagtgc ataatgccaa gacaaagccg cgggaggagc agtacaacag cacgtaccgt | 5460 |
| | gtggtcagcg tcctcaccgt cctgcaccaa gactggctga atggcaagga gtacaagtgc | 5520 |
| | aaggtctcca acaaagccct cccagccccc atcgagaaaa ccatctccaa agccaaaggg | 5580 |
| | cagccccgag aaccacaggt gtacaccctg cccccatccc gggatgagct gaccaagaac | 5640 |
| | caagttagcc tgacctgcct ggtcaaaggc ttctatccca gcgacatcgc cgtggagtgg | 5700 |
| | gagagcaatg ggcagccgga gaacaactac aagaccacgc ctcccgtgtt ggactccgac | 5760 |
| | ggctccttct tcctctactc caagctcacc gtggacaaga gcaggtggca gcaggggaac | 5820 |
| | gtcttctcat gctccgtgat gcatgaggct ctgcacaacc actacacgca gaagagcctc | 5880 |
| | tccctgtctc cgggttga | 5898 |
| rFVIIIFc-ELNN | ATRRYYLGAV ELSWDYMQSD LGELPVDARF PPRVPKSFPF NTSVVYKKTL FVEFTDHLFN | 60 |
| amino acid | IAKPRPPWMG LLGPTIQAEV YDTVVITLKN MASHPVSLHA VGVSYWKASE GAEYDDQTSQ | 120 |
| sequence, | REKEDDKVFP GGSHTYVWQV LKENGPMASD PLCLTYSYLS HVDLVKDLNS GLIGALLVCR | 180 |
| without signal | EGSLAKEKTQ TLHKFILLFA VFDEGKSWHS ETKNSLMQDR DAASARAWPK MHTVNGYVNR | 240 |
| peptide | SLPGLIGCHR KSVYWHVIGM GTTPEVHSIF LEGHTFLVRN HRQASLEISP ITFLTAQTLL | 300 |
| (SEQ ID NO: 3) | MDLGQFLLFC HISSHQHDGM EAYVKVDSCP EEPQLRMKNN EEAEDYDDDL TDSEMDVVRF | 360 |
| | DDDNSPSFIQ IRSVAKKHPK TWVHYIAAEE EDWDYAPLVL APDDRSYKSQ YLNNGPQRIG | 420 |
| | RKYKKVRFMA YTDETFKTRE AIQHESGILG PLLYGEVGDT LLIIFKNQAS RPYNIYPHGI | 480 |
| | TDVRPLYSRR LPKGVKHLKD FPILPGEIFK YKWTVTVEDG PTKSDPRCLT RYYSSFVNME | 540 |
| | RDLASGLIGP LLICYKESVD QRGNQIMSDK RNVILFSVFD ENRSWYLTEN IQRFLPNPAG | 600 |
| | VQLEDPEFQA SNIMHSINGY VFDSLQLSVC LHEVAYWYIL SIGAQTDFLS VFFSGYTFKH | 660 |
| | KMVYEDTLTL FPFSGETVFM SMENPGLWIL GCHNSDFRNR GMTALLKVSS CDKNTGDYYE | 720 |
| | DSYEDISAYL LSKNNAIEPR SFSQNGTSES ATPESGPGSE PATSGSETPG TSESATPESG | 780 |
| | PGSEPATSGS ETPGTSESAT PESGPGTSTE PSEGSAPGSP AGSPTSTEEG TSESATPESG | 840 |
| | PGSEPATSGS ETPGTSESAT PESGPGSPAG SPTSTEEGSP AGSPTSTEEG TSTEPSEGSA | 900 |
| | PGTSESATPE SGPGTSESAT PESGPGTSES ATPESGPGSE PATSGSETPG SEPATSGSET | 960 |
| | PGSPAGSPTS TEEGTSTEPS EGSAPGTSTE PSEGSAPGSE PATSGSETPG TSESATPESG | 1020 |
| | PGTSTEPSEG SAPASSEITR TTLQSDQEEI DYDDTISVEM KKEDFDIYDE DENQSPRSFQ | 1080 |
| | KKTRHYFIAA VERLWDYGMS SSPHVLRNRA QSGSVPQFKK VVFQEFTDGS FTQPLYRGEL | 1140 |
| | NEHLGLLGPY IRAEVEDNIM VTFRNQASRP YSFYSSLISY EEDQRQGAEP RKNFVKPNET | 1200 |
| | KTYFWKVQHH MAPTKDEFDC KAWAYFSDVD LEKDVHSGLI GPLLVCHTNT LNPAHGRQVT | 1260 |
| | VQEFALFFTI FDETKSWYFT ENMERNCRAP CNIQMEDPTF KENYRFHAIN GYIMDTLPGL | 1320 |
| | VMAQDQRIRW YLLSMGSNEN IHSIHFSGHV FTVRKKEEYK MALYNLYPGV FETVEMLPSK | 1380 |
| | AGIWRVECLI GEHLHAGMST LFLVYSNKCQ TPLGMASGHI RDFQITASGQ YGQWAPKLAR | 1440 |
| | LHYSGSINAW STKEPFSWIK VDLLAPMIIH GIKTQGARQK FSSLYISQFI IMYSLDGKKW | 1500 |
| | QTYRGNSTGT LMVFFGNVDS SGIKHNIFNP PIIARYIRLH PTHYSIRSTL RMELMGCDLN | 1560 |
| | SCSMPLGMES KAISDAQITA SSYFTNMFAT WSPSKARLHL QGRSNAWRPQ VNNPKEWLQV | 1620 |
| | DFQKTMKVTG VTTQGVKSLL TSMYVKEFLI SSSQDGHQWT LFFQNGKVKV FQGNQDSFTP | 1680 |
| | VVNSLDPPLL TRYLRIHPQS WVHQIALRME VLGCEAQDLY DKTHTCPPCP APELLGGPSV | 1740 |
| | FLFPPKPKDT LMISRTPEVT CVVVDVSHED PEVKFNWYVD GVEVHNAKTK PREEQYNSTY | 1800 |
| | RVVSVLTVLH QDWLNGKEYK CKVSNKALPA PIEKTISKAK GQPREPQVYT LPPSRDELTK | 1860 |
| | NQVSLTCLVK GFYPSDIAVE WESNGQPENN YKTTPPVLDS DGSFFLYSKL TVDKSRWQQG | 1920 |
| | NVFSCSVMHE ALHNHYTQKS LSLSPG | 1946 |
| rVWFFc-ELNN | MIPARFAGVL LALALILPGT LCAEGTRGRS STARCSLFGS DFVNTFDGSM YSFAGYCSYL | 60 |
| amino acid | LAGGCQKRSF SIIGDFQNGK RVSLSVYLGE FFDIHLFVNG TVTQGDQRVS MPYASKGLYL | 120 |
| sequence, | ETEAGYYKLS GEAYGFVARI DGSGNFQVLL SDRYFNKTCG LCGNFNIFAE DDFMTQEGTL | 180 |
| with signal | TSDPYDFANS WALSSGEQWC ERASPPSSSC NISSGEMQKG LWEQCQLLKS TSVFARCHPL | 240 |
| peptide and | VDPEPFVALC EKTLCECAGG LECACPALLE YARTCAQEGM VLYGWTDHSA CSPVCPAGME | 300 |
| D1D2 portion | YRQCVSPCAR TCQSLHINEM CQERCVDGCS CPEGQLLDEG LCVESTECPC VHSGKRYPPG | 360 |
| of VWF | TSLSRDCNTC ICRNSQWICS NEECPGECLV TGQSHFKSFD NRYFTFSGIC QYLLARDCQD | 420 |
| (SEQ ID NO: 4) | HSFSIVIETV QCADDRDAVC TRSVTVRLPG LHNSLVKLKH GAGVAMDGQD IQLPLLKGDL | 480 |
| | RIQHTVTASV RLSYGEDLQM DWDGRGRLLV KLSPVYAGKT CGLCGNYNGN QGDDFLTPSG | 540 |
| | LAEPRVEDFG NAWKLHGDCQ DLQKQHSDPC ALNPRMTRFS EEACAVLTSP TFEACHRAVS | 600 |
| | PLPYLRNCRY DVCSCSDGRE CLCGALASYA AACAGRGVRV AWREPGRCEL NCPKGQVYLQ | 660 |
| | CGTPCNLTCR SLSYPDEECN EACLEGCFCP PGLYMDERGD CVPKAQCPCY YDGEIFQPED | 720 |
| | IFSDHHTMCY CEDGFMHCTM SGVPGSLLPD AVLSSPLSHR SKRSLSCRPP MVKLVCPADN | 780 |
| | LRAEGLECTK TCQNYDLECM SMGCVSGCLC PPGMVRHENR CVALERCPCF HQGKEYAPGE | 840 |
| | TVKIGCNTCV CRDRKWNCTD HVCDATCSTI GMAHYLTFDG LKYLFPGECQ YVLVQDYCGS | 900 |
| | NPGTFRILVG NKGCSHPSVK CKKRVTILVE GGEIELFDGE VNVKRPMKDE THFEVVESGR | 960 |
| | YIILLLGKAL SVVWDRHLSI SVVLKQTYQE KVCGLCGNFD GIQNNDLTSS NLQVEEDPVD | 1020 |
| | FGNSWKVSSQ CADTRKVPLD SSPATCHNNI MKQTMVDSSC RILTSDVFQD CNKLVDPEPY | 1080 |
| | LDVCIYDTCS CESIGDCAAF CDTIAAYAHV CAQHGKVVTW RTATLCPQSC EERNLRENGY | 1140 |
| | EAEWRYNSCA PACQVTCQHP EPLACPVQCV EGCHAHCPPG KILDELLQTC VDPEDCPVCE | 1200 |
| | VAGRRFASGK KVTLNPSDPE HCQICHCDVV NLTCEACQEP GTSESATPES GPGSEPATSG | 1260 |
| | SETPGTSESA TPESGPGSEP ATSGSETPGT SESATPESGP GTSTEPSEGS APGSPAGSPT | 1320 |
| | STEEGTSESA TPESGPGSEP ATSGSETPGT SESATPESGP GSPAGSPTST EEGSPAGSPT | 1380 |
| | STEEGASSDK NTGDYYEDSY EDISAYLLSK NNAIEPRSFS DKTHTCPPCP APELLGGPSV | 1440 |
| | FLFPPKPKDT LMISRTPEVT CVVVDVSHED PEVKFNWYVD GVEVHNAKTK PREEQYNSTY | 1500 |
| | RVVSVLTVLH QDWLNGKEYK CKVSNKALPA PIEKTISKAK GQPREPQVYT LPPSRDELTK | 1560 |
| | NQVSLTCLVK GFYPSDIAVE WESNGQPENN YKTTPPVLDS DGSFFLYSKL TVDKSRWQQG | 1620 |
| | NVFSCSVMHE ALHNHYTQKS LSLSPG | 1646 |
| rVWFFc-ELNN | atgattcctg ccagatttgc cggggtgctg cttgctctgg ccctcatttt gccagggacc | 60 |
| nucleotide | ctttgtgcag aaggaactcg cggcaggtca tccacggccc gatgcagcct tttcggaagt | 120 |
| sequence | gacttcgtca acacctttga tgggagcatg tacagctttg cgggatactg cagttacctc | 180 |
| (SEQ ID NO: 5) | ctggcagggg gctgccagaa acgctccttc tcgattattg gggacttcca gaatggcaag | 240 |
| | agagtgagcc tctccgtgta tcttggggaa ttttttgaca tccatttgtt tgtcaatggt | 300 |
| | accgtgacac agggggacca aagagtctcc atgccctatg cctccaaagg gctgtatcta | 360 |
| | gaaactgagg ctgggtacta caagctgtcc ggtgaggcct atggctttgt ggccaggatc | 420 |
| | gatggcagcg gcaactttca agtcctgctg tcagacagat acttcaacaa gacctgcggg | 480 |
| | ctgtgtggca actttaacat ctttgctgaa gatgacttta tgacccaaga agggaccttg | 540 |
| | acctcggacc cttatgactt tgccaactca tgggctctga gcagtggaga acagtggtgt | 600 |
| | gaacgggcat ctcctcccag cagctcatgc aacatctcct ctggggaaat gcagaagggc | 660 |
| | ctgtgggagc agtgccagct tctgaagagc acctcggtgt ttgcccgctg ccaccctctg | 720 |
| | gtggaccccg agccttttgt ggccctgtgt gagaagactt tgtgtgagtg tgctgggggg | 780 |
| | ctggagtgcg cctgccctgc cctcctggag tacgcccgga cctgtgccca ggagggaatg | 840 |
| | gtgctgtacg gctggaccga ccacagcgcg tgcagcccag tgtgccctgc tggtatggag | 900 |
| | tataggcagt gtgtgtcccc ttgcgccagg acctgccaga gcctgcacat caatgaaatg | 960 |
| | tgtcaggagc gatgcgtgga tggctgcagc tgccctgagg gacagctcct ggatgaaggc | 1020 |
| | ctctgcgtgg agagcaccga gtgtccctgc gtgcattccg gaaagcgcta ccctcccggc | 1080 |
| | acctccctct ctcgagactg caacacctgc atttgccgaa acagccagtg gatctgcagc | 1140 |
| | aatgaagaat gtccagggga gtgccttgtc actggtcaat cccacttcaa gagctttgac | 1200 |
| | aacagatact tcaccttcag tgggatctgc cagtacctgc tggcccggga ttgccaggac | 1260 |
| | cactccttct ccattgtcat tgagactgtc cagtgtgctg atgaccgcga cgctgtgtgc | 1320 |
| | acccgctccg tcaccgtccg gctgcctggc ctgcacaaca gccttgtgaa actgaagcat | 1380 |
| | ggggcaggag ttgccatgga tggccaggac atccagctcc ccctcctgaa aggtgacctc | 1440 |
| | cgcatccagc atacagtgac ggcctccgtg cgcctcagct acggggagga cctgcagatg | 1500 |
| | gactgggatg gccgcgggag gctgctggtg aagctgtccc ccgtctatgc cgggaagacc | 1560 |
| | tgcggcctgt gtgggaatta caatggcaac cagggcgacg acttccttac cccctctggg | 1620 |
| | ctggcggagc cccgggtgga ggacttcggg aacgcctgga agctgcacgg ggactgccag | 1680 |
| | gacctgcaga agcagcacag cgatccctgc gccctcaacc cgcgcatgac caggttctcc | 1740 |
| | gaggaggcgt gcgcggtcct gacgtccccc acattcgagg cctgccatcg tgccgtcagc | 1800 |
| | ccgctgccct acctgcggaa ctgccgctac gacgtgtgct cctgctcgga cggccgcgag | 1860 |
| | tgcctgtgcg gcgccctggc cagctatgcc gcggcctgcg cggggagagg cgtgcgcgtc | 1920 |
| | gcgtggcgcg agccaggccg ctgtgagctg aactgcccga aaggccaggt gtacctgcag | 1980 |
| | tgcgggaccc cctgcaacct gacctgccgc tctctctctt acccggatga ggaatgcaat | 2040 |
| | gaggcctgcc tggagggctg cttctgcccc ccagggctct acatggatga gaggggggac | 2100 |
| | tgcgtgccca aggcccagtg cccctgttac tatgacggtg agatcttcca gccagaagac | 2160 |
| | atcttctcag accatcacac catgtgctac tgtgaggatg gcttcatgca ctgtaccatg | 2220 |
| | agtggagtcc ccggaagctt gctgcctgac gctgtcctca gcagtcccct gtctcatcgc | 2280 |
| | agcaaaagga gcctatcctg tcggcccccc atggtcaagc tggtgtgtcc cgctgacaac | 2340 |
| | ctgcgggctg aagggctcga gtgtaccaaa acgtgccaga actatgacct ggagtgcatg | 2400 |
| | agcatgggct gtgtctctgg ctgcctctgc cccccgggca tggtccggca tgagaacaga | 2460 |
| | tgtgtggccc tcgaaaggtg tccctgcttc catcagggca aggagtatgc ccctggagaa | 2520 |
| | acagtgaaga ttggctgcaa cacttgtgtc tgtcgggacc ggaagtggaa ctgcacagac | 2580 |
| | catgtgtgtg atgccacgtg ctccacgatc ggcatggccc actacctcac cttcgacggg | 2640 |
| | ctcaaatacc tgttccccgg ggagtgccag tacgttctgg tgcaggatta ctgcggcagt | 2700 |
| | aaccctggga cctttcggat cctagtgggg aataagggat gcagccaccc ctcagtgaaa | 2760 |
| | tgcaagaaac gggtcaccat cctggtggag ggaggagaga ttgagctgtt tgacggggag | 2820 |
| | gtgaatgtga agaggcccat gaaggatgag actcactttg aggtggtgga gtctggccgg | 2880 |
| | tacatcattc tgctgctggg caaagccctc tccgtggtct gggaccgcca cctgagcatc | 2940 |
| | tccgtggtcc tgaagcagac ataccaggag aaagtgtgtg gcctgtgtgg gaattttgat | 3000 |
| | ggcatccaga acaatgacct caccagcagc aacctccaag tggaggaaga ccctgtggac | 3060 |
| | tttgggaact cctggaaagt gagctcgcag tgtgctgaca ccagaaaagt gcctctggac | 3120 |
| | tcatcccctg ccacctgcca taacaacatc atgaagcaga cgatggtgga ttcctcctgt | 3180 |
| | agaatcctta ccagtgacgt cttccaggac tgcaacaagc tggtggaccc cgagccatat | 3240 |
| | ctggatgtct gcatttacga cacctgctcc tgtgagtcca ttggggactg cgccgcattc | 3300 |
| | tgcgacacca ttgctgccta tgcccacgtg tgtgcccagc atggcaaggt ggtgacctgg | 3360 |
| | aggacggcca cattgtgccc ccagagctgc gaggagagga atctccggga gaacgggtat | 3420 |
| | gaggctgagt ggcgctataa cagctgtgca cctgcctgtc aagtcacgtg tcagcaccct | 3480 |
| | gagccactgg cctgccctgt gcagtgtgtg gagggctgcc atgcccactg ccctccaggg | 3540 |
| | aaaatcctgg atgagctttt gcagacctgc gttgaccctg aagactgtcc agtgtgtgag | 3600 |
| | gtggctggcc ggcgttttgc ctcaggaaag aaagtcacct tgaatcccag tgaccctgag | 3660 |
| | cactgccaga tttgccactg tgatgttgtc aacctcacct gtgaagcctg ccaggagccg | 3720 |
| | ggtacatcag agagcgccac ccctgaaagt ggtcccggga gcgagccagc cacatctggg | 3780 |
| | tcggaaacgc caggcacatc cgagtctgca actcccgagt ccggacctgg ctccgagcct | 3840 |
| | gccactagcg gctccgagac tccgggaact tccgagagcg ctacaccaga aagcggaccc | 3900 |
| | ggaaccagta ccgaacctag cgagggctct gctccgggca gcccagccgg ctctcctaca | 3960 |
| | tccacggagg agggcacttc cgaatccgcc accccggagt cagggccagg atctgaaccc | 4020 |
| | gctacctcag gcagtgagac gccaggaacg agcgagtccg ctacaccgga gagtgggcca | 4080 |
| | gggagccctg ctggatctcc tacgtccact gaggaagggt caccagcggg ctcgcccacc | 4140 |
| | agcactgaag aaggtgcctc gtctgacaag aacactggtg attattacga ggacagttat | 4200 |
| | gaagatattt cagcatactt gctgagtaaa aacaatgcca ttgaaccaag aagcttctct | 4260 |
| | gacaaaactc acacatgccc accgtgccca gctccagaac tcctgggcgg accgtcagtc | 4320 |
| | ttcctcttcc ccccaaaacc caaggacacc ctcatgatct cccggacccc tgaggtcaca | 4380 |
| | tgcgtggtgg tggacgtgag ccacgaagac cctgaggtca agttcaactg gtatgtggac | 4440 |
| | ggcgtggaag tgcataatgc caagacaaag ccgcgggagg agcagtacaa cagcacgtac | 4500 |
| | cgtgtggtca gcgtcctcac cgtcctgcac caagactggc tgaatggcaa ggagtacaag | 4560 |
| | tgcaaggtct ccaacaaagc cctcccagcc cccatcgaga aaaccatctc caaagccaaa | 4620 |
| | gggcagcccc gagaaccaca ggtgtacacc ctgcccccat cccgggatga gctgaccaag | 4680 |
| | aaccaagtta gcctgacctg cctggtcaaa ggcttctatc ccagcgacat cgccgtggag | 4740 |
| | tgggagagca atgggcagcc ggagaacaac tacaagacca cgcctcccgt gttggactcc | 4800 |
| | gacggctcct tcttcctcta ctccaagctc accgtggaca agagcaggtg gcagcagggg | 4860 |
| | aacgtcttct catgctccgt gatgcatgag gctctgcaca accactacac gcagaagagc | 4920 |
| | ctctccctgt ctccgggttg a | 4941 |
| rVWF-ELNN-Fc | SLSCRPPMVK LVCPADNLRA EGLECTKTCQ NYDLECMSMG CVSGCLCPPG MVRHENRCVA | 60 |
| amino acid | LERCPCFHQG KEYAPGETVK IGCNTCVCRD RKWNCTDHVC DATCSTIGMA HYLTFDGLKY | 120 |
| sequence, | LFPGECQYVL VQDYCGSNPG TFRILVGNKG CSHPSVKCKK RVTILVEGGE IELFDGEVNV | 180 |
| without signal | KRPMKDETHF EVVESGRYII LLLGKALSVV WDRHLSISVV LKQTYQEKVC GLCGNFDGIQ | 240 |
| peptide or | NNDLTSSNLQ VEEDPVDFGN SWKVSSQCAD TRKVPLDSSP ATCHNNIMKQ TMVDSSCRIL | 300 |
| D1D2 portion | TSDVFQDCNK LVDPEPYLDV CIYDTCSCES IGDCAAFCDT IAAYAHVCAQ HGKVVTWRTA | 360 |
| of VWF | TLCPQSCEER NLRENGYEAE WRYNSCAPAC QVTCQHPEPL ACPVQCVEGC HAHCPPGKIL | 420 |
| (SEQ ID NO: 6) | DELLQTCVDP EDCPVCEVAG RRFASGKKVT LNPSDPEHCQ ICHCDVVNLT CEACQEPGTS | 480 |
| | ESATPESGPG SEPATSGSET PGTSESATPE SGPGSEPATS GSETPGTSES ATPESGPGTS | 540 |
| | TEPSEGSAPG SPAGSPTSTE EGTSESATPE SGPGSEPATS GSETPGTSES ATPESGPGSP | 600 |
| | AGSPTSTEEG SPAGSPISTE EGASSDKNTG DYYEDSYEDI SAYLLSKNNA IEPRSFSDKT | 660 |
| | HTCPPCPAPE LLGGPSVFLF PPKPKDTLMI SRTPEVTCVV VDVSHEDPEV KFNWYVDGVE | 720 |
| | VHNAKTKPRE EQYNSTYRVV SVLTVLHQDW LNGKEYKCKV SNKALPAPIE KTISKAKGQP | 780 |
| | REPQVYTLPP SRDELTKNQV SLTCLVKGFY PSDIAVEWES NGQPENNYKT TPPVLDSDGS | 840 |
| | FFLYSKLTVD KSRWQQGNVF SCSVMHEALH NHYTQKSLSL SPG | 883 |
| Mature human | ATRRYYLGAV ELSWDYMQSD LGELPVDARF PPRVPKSFPF NTSVVYKKTL FVEFTDHLFN | 60 |
| FVIII | IAKPRPPWMG LLGPTIQAEV YDTVVITLKN MASHPVSLHA VGVSYWKASE GAEYDDQTSQ | 120 |
| (SEQ ID NO: 7) | REKEDDKVFP GGSHTYVWQV LKENGPMASD PLCLTYSYLS HVDLVKDLNS GLIGALLVCR | 180 |
| | EGSLAKEKTQ TLHKFILLFA VFDEGKSWHS ETKNSLMQDR DAASARAWPK MHTVNGYVNR | 240 |
| | SLPGLIGCHR KSVYWHVIGM GTTPEVHSIF LEGHTFLVRN HRQASLEISP ITFLTAQTLL | 300 |
| | MDLGQFLLFC HISSHQHDGM EAYVKVDSCP EEPQLRMKNN EEAEDYDDDL TDSEMDVVRF | 360 |
| | DDDNSPSFIQ IRSVAKKHPK TWVHYIAAEE EDWDYAPLVL APDDRSYKSQ YLNNGPQRIG | 420 |
| | RKYKKVRFMA YTDETFKTRE AIQHESGILG PLLYGEVGDT LLIIFKNQAS RPYNIYPHGI | 480 |
| | TDVRPLYSRR LPKGVKHLKD FPILPGEIFK YKWTVTVEDG PTKSDPRCLT RYYSSFVNME | 540 |
| | RDLASGLIGP LLICYKESVD QRGNQIMSDK RNVILFSVFD ENRSWYLTEN IQRFLPNPAG | 600 |
| | VQLEDPEFQA SNIMHSINGY VFDSLQLSVC LHEVAYWYIL SIGAQTDFLS VFFSGYTFKH | 660 |
| | KMVYEDTLTL FPFSGETVFM SMENPGLWIL GCHNSDFRNR GMTALLKVSS CDKNTGDYYE | 720 |
| | DSYEDISAYL LSKNNAIEPR SFSQNSRHPS TRQKQFNATT IPENDIEKTD PWFAHRTPMP | 780 |
| | KIQNVSSSDL LMLLRQSPTP HGLSLSDLQE AKYETFSDDP SPGAIDSNNS LSEMTHFRPQ | 840 |
| | LHHSGDMVFT PESGLQLRLN EKLGTTAATE LKKLDFKVSS TSNNLISTIP SDNLAAGTDN | 900 |
| | TSSLGPPSMP VHYDSQLDTT LFGKKSSPLT ESGGPLSLSE ENNDSKLLES GLMNSQESSW | 960 |
| | GKNVSSTESG RLFKGKRAHG PALLTKDNAL FKVSISLLKT NKTSNNSATN RKTHIDGPSL | 1020 |
| | LIENSPSVWQ NILESDTEFK KVTPLIHDRM LMDKNATALR LNHMSNKTTS SKNMEMVQQK | 1080 |
| | KEGPIPPDAQ NPDMSFFKML FLPESARWIQ RTHGKNSLNS GQGPSPKQLV SLGPEKSVEG | 1140 |
| | QNFLSEKNKV VVGKGEFTKD VGLKEMVFPS SRNLFLTNLD NLHENNTHNQ EKKIQEEIEK | 1200 |
| | KETLIQENVV LPQIHTVTGT KNFMKNLFLL STRQNVEGSY DGAYAPVLQD FRSLNDSTNR | 1260 |
| | TKKHTAHFSK KGEEENLEGL GNQTKQIVEK YACTTRISPN TSQQNFVTQR SKRALKQFRL | 1320 |
| | PLEETELEKR IIVDDTSTQW SKNMKHLTPS TLTQIDYNEK EKGAITQSPL SDCLTRSHSI | 1380 |
| | PQANRSPLPI AKVSSFPSIR PIYLTRVLFQ DNSSHLPAAS YRKKDSGVQE SSHFLQGAKK | 1440 |
| | NNLSLAILTL EMTGDQREVG SLGTSATNSV TYKKVENTVL PKPDLPKTSG KVELLPKVHI | 1500 |
| | YQKDLFPTET SNGSPGHLDL VEGSLLQGTE GAIKWNEANR PGKVPFLRVA TESSAKTPSK | 1560 |
| | LLDPLAWDNH YGTQIPKEEW KSQEKSPEKT AFKKKDTILS LNACESNHAI AAINEGQNKP | 1620 |
| | EIEVTWAKQG RTERLCSQNP PVLKRHQREI TRTTLQSDQE EIDYDDTISV EMKKEDFDIY | 1680 |
| | DEDENQSPRS FQKKTRHYFI AAVERLWDYG MSSSPHVLRN RAQSGSVPQF KKVVFQEFTD | 1740 |
| | GSFTQPLYRG ELNEHLGLLG PYIRAEVEDN IMVTERNQAS RPYSFYSSLI SYEEDQRQGA | 1800 |
| | EPRKNFVKPN ETKTYFWKVQ HHMAPTKDEF DCKAWAYFSD VDLEKDVHSG LIGPLLVCHT | 1860 |
| | NTLNPAHGRQ VTVQEFALFF TIFDETKSWY FTENMERNCR APCNIQMEDP TFKENYRFHA | 1920 |
| | INGYIMDTLP GLVMAQDQRI RWYLLSMGSN ENIHSIHFSG HVFTVRKKEE YKMALYNLYP | 1980 |
| | GVFETVEMLP SKAGIWRVEC LIGEHLHAGM STLFLVYSNK CQTPLGMASG HIRDFQITAS | 2040 |
| | GQYGQWAPKL ARLHYSGSIN AWSTKEPFSW IKVDLLAPMI IHGIKTQGAR QKFSSLYISQ | 2100 |
| | FIIMYSLDGK KWQTYRGNST GTLMVFFGNV DSSGIKHNIF NPPIIARYIR LHPTHYSIRS | 2160 |
| | TLRMELMGCD LNSCSMPLGM ESKAISDAQI TASSYFTNMF ATWSPSKARL HLQGRSNAWR | 2220 |
| | PQVNNPKEWL QVDFQKTMKV TGVTTQGVKS LLTSMYVKEF LISSSQDGHQ WTLFFQNGKV | 2280 |
| | KVFQGNQDSF TPVVNSLDPP LLTRYLRIHP QSWVHQIALR MEVLGCEAQD LY | 2332 |
| ELNN | GTSESATPES GPGSEPATSG SETPGTSESA TPESGPGSEP ATSGSETPGT SESATPESGP | 60 |
| polypeptide | GTSTEPSEGS APGSPAGSPT STEEGTSESA TPESGPGSEP ATSGSETPGT SESATPESGP | 120 |
| AE288 | GSPAGSPTST EEGSPAGSPT STEEGTSTEP SEGSAPGTSE SATPESGPGT SESATPESGP | 180 |
| (SEQ ID NO: 8) | GTSESATPES GPGSEPATSG SETPGSEPAT SGSETPGSPA GSPTSTEEGT STEPSEGSAP | 240 |
| | GTSTEPSEGS APGSEPATSG SETPGTSESA TPESGPGTST EPSEGSAP | 288 |
| ELNN | TSESATPESG PGSEPATSGS ETPGTSESAT PESGPGSEPA TSGSETPGTS ESATPESGPG | 60 |
| polypeptide | TSTEPSEGSA PGSPAGSPTS TEEGTSESAT PESGPGSEPA TSGSETPGTS ESATPESGPG | 120 |
| AE144_5A | SPAGSPTSTE EGSPAGSPTS TEEG | 144 |
| (SEQ ID NO: 9) | | |
| a2 Linker of | DKNTGDYYED SYEDISAYLL SKNNAIEPRS FS | 32 |
| chimeric | | |
| protein | | |
| (SEQ ID NO: 10) | | |

1. A method for determining efanesoctocog alfa dosing information for an individual subject, the method comprising
receiving information specific to the subject and calculating the efanesoctocog alfa dosing information using a software-based system,
wherein the system is programmed to implement a one-compartment efanesoctocog alfa popPK model,
wherein the efanesoctocog alfa popPK model comprises body weight as a covariate, and wherein the efanesoctocog alfa popPK model does not comprise the level of VWF or level of hematocrit as covariates.

2. The method of claim 1, further comprising outputting, by the software-based system, the dosing information for the subject.

3. The method of claim 2, further comprising outputting, by the software-based system, a suggested dose regimen.

4. A method of estimating individualized subject efanesoctocog alfa PK information, the method comprising:
(a) receiving subject information by a software-based system comprising an efanesoctocog alfa popPK model,
(b) estimating, by the software-based system, individualized subject efanesoctocog alfa PK information using the efanesoctocog alfa popPK model and the received information, and
(c) outputting, by the software-based system, the individualized subject efanesoctocog alfa PK information,
wherein the efanesoctocog alfa popPK model comprises body weight as a covariate, and wherein the efanesoctocog alfa popPK model does not comprise the level of VWF or level of hematocrit as covariates.

5. The method of claim 4, wherein the subject information includes a baseline FVIII activity level for the subject.

6. (canceled)

7. The method of claim 1, wherein the system is programmed to implement a one-compartment efanesoctocog alfa popPK model comprising body weight as covariates to calculate the dosing information, and wherein the efanesoctocog alfa popPK model does not comprise the level of VWF or hematocrit as covariates.

8. The method of claim 4, further comprising selecting a dosing regimen based on the PK information.

9. The method of claim 8, further comprising administering the efanesoctocog alfa to the subject according to the selected dosing regimen.

10. The method of claim 1, wherein the dosing information comprises estimated or predicted FVIII activity levels over time after administration of the efanesoctocog alfa.

11. The method of claim 8, wherein the dosing regimen is a prophylaxis regimen, an on-demand regimen, or is for perioperative management of bleeding.

12. The method of claim 1, wherein the efanesoctocog alfa popPK model comprises whether the subject self-reports as Asian as a covariate.

13. The method of claim 1, wherein the efanesoctocog alfa popPK model is the efanesoctocog alfa popPK model [A].

14. The method of claim 13, wherein the estimate of typical clearance (TVCL) in the efanesoctocog alfa popPK model [A] is 0.433 dL/h, the estimate of typical volume (TVV) in the efanesoctocog alfa popPK model [A] is 30.2 dL, the variability on clearance from central compartment (η₁) in the efanesoctocog alfa popPK model [A] is 0.0354, and the variability on volume of central compartment (η₂) in the efanesoctocog alfa popPK model [A] is 0.0209.

15. A method of treating hemophilia A in a subject in need thereof, comprising administering to the subject a dose regimen selected according to claim 1.

16. A device or system comprising a processor configured to provide dosing or PK information according to the method of claim 1.

17. A data processing apparatus, device, or system comprising a processor configured to implement a one-compartment efanesoctocog alfa popPK model, wherein the efanesoctocog alfa popPK model comprises body weight as a covariate, and wherein the efanesoctocog alfa popPK model does not comprise the level of VWF or level of hematocrit as covariates.

18. (canceled)

19. A computer program comprising instructions which, when the program is executed by a computer, cause the computer to carry out the method of claim 1.

20. A computer program comprising instructions which, when the program is executed by a computer, cause the computer to implement a one-compartment efanesoctocog alfa popPK model, wherein the efanesoctocog alfa popPK model comprises body weight as a covariate, and wherein the efanesoctocog alfa popPK model does not comprise the level of VWF or level of hematocrit as covariates.

21. (canceled)

22. A computer-readable medium comprising instructions which, when executed by a computer, cause the computer to provide carry out the method of claim 1.

23. A computer-readable medium comprising instructions which, when executed by a computer, cause the computer to implement a one-compartment efanesoctocog alfa popPK model, wherein the efanesoctocog alfa popPK model comprises body weight as a covariate, and wherein the efanesoctocog alfa popPK model does not comprise the level of VWF or level of hematocrit as covariates.

24. (canceled)
